(19) United States
(12) Patent Application Publication
(10) Pub. No.: US 2018/0372817 A1
(43) Pub. Date: Dec. 27, 2018

(54) Flexible Coil for Magnetic Resonance Imaging

(71) Applicant: The Regents of the University of California, Oakland, CA (US)

(72) Inventors: Yahya Rahmat-Samii, Sherman Oaks, CA (US); Daisong Zhang, Los Angeles, CA (US)

(73) Assignee: The Regents of the University of California, Oakland, CA (US)

(21) Appl. No.: 16/018,704

(22) Filed: Jun. 26, 2018

Related U.S. Application Data
(60) Provisional application No. 62/525,590, filed on Jun. 27, 2017.

(51) Int. Cl. G01R 33/34; A61B 5/055

(52) U.S. Cl. CPC G01R 33/34084; A61B 5/055

(57) ABSTRACT

Radiofrequency (RF) coil elements and arrays for magnetic resonance imaging (MRI) in accordance with embodiments of the invention are disclosed. In one embodiment of the invention, a coil element for MRI comprises a fabric; a coil pattern fixed to the fabric, the coil pattern comprising cut conductive cloth and being capable of receiving radiofrequency (RF) signals from a human body; a tuning component attached to the coil pattern and capable of adjusting frequency of the coil element; and an electrical component coupled with the coil pattern, the electrical component capable of transmitting data received at the coil element.

### CROSS-REFERENCE TO RELATED APPLICATION(S)

The current application claims priority to U.S. Provisional Patent Application Ser. No. 62/525,590 entitled “Flexible Coil for Magnetic Resonance Imaging,” filed Jun. 27, 2017, the disclosure of which is herein incorporated by reference in its entirety.

### FIELD OF THE INVENTION

This invention generally relates to a flexible coil system for use in magnetic resonance imaging (MRI). More particularly, this invention relates to flexible radio frequency (RF) coil array systems utilizing electro-textiles for MRI imaging.

### BACKGROUND

Magnetic resonance imaging (MRI) was developed in the 1970s, and began to be widely used clinically in 1980s. Today, more than 20,000 MRI scanners are available worldwide, and more than 70 million magnetic resonance (MR) scans are performed every year. MRI is considered to be one of the most powerful imaging methods available. One advantage of MRI modality is that it is non-invasive, and patients are not exposed to ionizing radiation. Another advantage is that MR is capable of scanning arbitrary scan planes and 3D volumes. Further, MR provides quantitative information about the imaging area, making possible the prediction of more detailed information.

MRI technology is used in the medical field to evaluate and diagnose conditions of the human body. The MRI technique is used to form anatomical and physiological images of the body. Using magnetic fields, radio waves, and field gradients, MRI scanners can generate images of body organs. A patient may be positioned within an MRI scanner, which transmits a radio frequency to the body area to be imaged. The patient's tissues emit a radio frequency signal, which is measured by a radio frequency (RF) coil. The RF coil acts as a receiver, and sometimes a transmitter, of the RF signals from and to the patient's body, and transmits the data for image generation.

RF coils for use in MRI imaging are typically specialized for the type of scan required. Volume coils provide homogenous RF signals across a large volume, and are used to perform whole-body scans as well as the head and other extremities. Surface coils, on the other hand, are designed to provide high RF sensitivity over a small region of interest, and can be size-optimized for a specific anatomical region.

Stroke is a condition that occurs frequently and is a leading cause of death worldwide. A stroke occurs when blood supply to the brain is interrupted or significantly reduced, resulting in deprivation of oxygen and nutrients to brain tissue. As a medical emergency, stroke requires prompt treatment. Stroke cases are often related to carotid artery diseases. The carotid arteries are the two major vessels near the neck. Pieces of plaque can break free, travel to the brain, and block blood vessels that supply blood to the brain. The carotid arteries thus provide a suitable location for imaging arterial plaques and monitoring disease progression, and MRI imaging of the carotid arteries can be a pivotal step in stroke prevention and treatment.

### SUMMARY OF THE INVENTION

Flexible RF coils for use in MRI imaging, in accordance with various embodiments of the invention are disclosed.

In one embodiment of the invention, a coil element for magnetic resonance imaging (MRI) comprises a fabric; a coil pattern fixed to the fabric, the coil pattern comprising cut conductive cloth and being capable of receiving radiofrequency (RF) signals from a human body; a tuning component attached to the coil pattern and capable of adjusting frequency of the coil element; and an electrical component coupled with the coil pattern, the electrical component capable of transmitting data received at the coil element.

In a further embodiment, the conductive cloth has a conductivity of at least 105 S/m, at a frequency of 120 MHz.

In another embodiment, the conductive cloth has a resistivity between 0.05 Ω/sq and 1 Ω/sq.

In a yet further embodiment, the conductive cloth is formed with comprises a non-magnetic and smooth surface.

In yet another embodiment, the electrical and mechanical properties of the conductive cloth are stable at a temperature of at least 200 degrees Celsius.

In a still further embodiment, the coil pattern is fixed to the fabric using by an adhesive.

In still another embodiment, the tuning component and the electrical component are soldered to the coil pattern.

In a further additional embodiment, the tuning component is selected from the group consisting of a varactor and variable capacitor.

A coil array for magnetic resonance imaging (MRI), according to another additional embodiment, comprises a first coil element, a second coil element, and an insulating sheet disposed between the first and second coil elements. The first and second coil elements may be disposed such that the first coil pattern overlaps with the second coil pattern. The first coil element may comprise a first fabric; and a first coil pattern fixed to the first fabric, the first coil pattern comprising a flexible conductive material and being capable of receiving radiofrequency (RF) signals from a human body. The second coil element may comprise a second fabric; and a second coil pattern fixed to the second fabric, the second coil pattern comprising a flexible conductive material and being capable of receiving radiofrequency (RF) signals from a human body.

In another further embodiment, one of the first and second coil patterns comprises conductive thread embroidered into the fabric.

In still another further embodiment, one of the first and second coil patterns comprises cut conductive cloth.

In a still yet further embodiment, the amount of overlap between the first and second coil patterns is configured to minimize mutual coupling between the first and second coil elements.

In still yet another embodiment, the amount of overlap between the first and second coil patterns is configured to maximize signal-to-noise ratio of the coil array.

In a further embodiment again, the insulating sheet is fixed to the first and second coil elements.

In another embodiment again, one of the first and second coil elements comprises a tuning component coupled with the coil pattern and capable of adjusting frequency of the coil array.

In a yet further embodiment again, the coil array further comprises a third coil element, including a third fabric; and a third coil pattern fixed to the third fabric, the third coil pattern comprising a flexible conductive material and being capable of receiving radiofrequency (RF) signals from a human body. The first, second and third coil elements may be disposed such that the third coil pattern first coil pattern overlaps with at least one of the first and second coil patterns.

In yet another embodiment again, the first and second coil elements are configured to conform to an anatomical region of the human body.

In a still further embodiment again, the coil array further comprises a barrier sheet covering the first and second coil elements.

In still another embodiment again, the coil array further comprises a flexible case within which the first and second coil elements are disposed.

In a further additional embodiment again, the flexible case comprises stretchable material.

### BRIEF DESCRIPTION OF THE DRAWINGS

FIG. 1 illustrates a 3T MRI scanner and an RF coil around a patient's neck.

FIG. 2 is a diagram of a coil element in accordance with certain embodiments of the invention.

FIG. 3 illustrates parts of a coil element in accordance with certain embodiments of the invention.

FIG. 4A shows examples of electro-textile threads used in accordance with certain embodiments of the invention.

FIG. 4B shows front and back views of an RF coil in accordance with certain embodiments of the invention.

FIG. 4C shows an example of conductive cloth and a coil element created using that conductive cloth, in accordance with certain embodiments of the invention.

FIG. 5 illustrates a front view of a coil array in accordance with certain embodiments of the invention.

FIG. 6 illustrates a side view of a multi-layer coil array in accordance with certain embodiments of the invention.

FIG. 7A shows a front view of a coil array in accordance with certain embodiments of the invention.

FIG. 7B is a diagram of a side view of a coil array structure in accordance with certain embodiments of the invention.

FIG. 7C shows simulated and measured S₁₁ and S₁₂ of the coil array of FIG. 7A.

FIG. 8A shows a first view of a flexible coil case in accordance with certain embodiments of the invention.

FIG. 8B shows a second view of a flexible coil case in accordance with certain embodiments of the invention.

FIG. 8C shows a close-up view of a flexible coil case in accordance with certain embodiments of the invention.

FIG. 9 shows a flexible coil case in relaxed and stretched positions in accordance with certain embodiments of the invention.

FIG. 10 shows a model wearing a flexible coil case in accordance with certain embodiments of the invention.

FIG. 11 shows examples of RF coils in accordance with certain embodiments of the invention.

FIG. 12A shows an example of an RF coil in accordance with certain embodiments of the invention.

FIG. 12B shows the H₁ field distribution, on the surface of the human neck with an input power of 1 W, of the RF coil of FIG. 12A.

FIG. 12C shows parameter values of the RF coil shown in FIG. 12A.

FIG. 13A shows tunable circuitry of a coil element in accordance with certain embodiments of the invention.

FIG. 13B shows an example of a coil element with tuning circuitry in accordance with certain embodiments of the invention.

FIG. 13C shows the measured S₁₁ of the RF coil element of FIG. 13B.

FIG. 14 shows an RF coil and its parameter values in accordance with certain embodiments of the invention.

FIG. 15 shows S₁₁ measurements for an RF coil in accordance with certain embodiments of the invention.

FIG. 16A shows a method to calculate the effective conductivity of an electro-textile pattern in accordance with certain embodiments of the invention.

FIG. 16B shows examples of microstrip lines used to calculate the effective conductivity of an electro-textile pattern in accordance with certain embodiments of the invention.

FIG. 17A shows simulated and measured S₁₁ of an RF coil in accordance with certain embodiments of the invention.

FIG. 17B shows simulated S₁₁ measurements for coil elements of varying diameter d and the touch resistance R_{touch} in accordance with certain embodiments of the invention.

FIG. 18A shows H₁ distribution results for an RF coil in accordance with certain embodiments of the invention.

FIG. 18B shows SAR distribution results for an RF coil in accordance with certain embodiments of the invention.

FIG. 18C shows a comparison of H₁ distribution results for an RF coil conformed to cylinders of varying diameters in accordance with certain embodiments of the invention.

FIG. 19 shows an example of unloaded and loaded quality factor measurement setups for evaluating the performance of RF coils in accordance with certain embodiments of the invention.

FIG. 20 shows an RF coil array integrated with an MRI scanner in accordance with certain embodiments of the invention.

FIG. 21 shows an RF coil array with a phantom placed in an MRI scanner for performance testing in accordance with certain embodiments of the invention.

FIG. 22 is an axial plane image of a homogenous cylindrical phantom taken with a 3T MRI scanner using an RF coil in accordance with certain embodiments of the invention.

FIG. 23 is an axial plane image of a resolution phantom taken with a 3T MRI scanner using an RF coil in accordance with certain embodiments of the invention.

FIG. 24 is an axial plane image of a beef phantom taken with a 3T MRI scanner using an RF coil in accordance with certain embodiments of the invention.

FIG. 25 illustrates an RF coil in communication with a processing system in accordance with certain embodiments of the invention.

### DETAILED DESCRIPTION

Turning now to the drawings, flexible RF coils for use in MRI imaging are illustrated. In many embodiments of the invention, highly conductive electro-textiles can enable increased RF sensitivity within a specific region to improve the ability of an MRI machine to image the region. The signal-to-noise ratio (SNR) of surface RF coils is highly sensitive to the placement of the coil relative to the imaged tissue, and when a coil is placed with greater proximity and conformance to the imaged tissue, an increase in signal sensitivity is expected. Thus, coil flexibility can provide increased SNR and thus higher quality MRI images. In several embodiments of the invention, tunable circuitry is utilized to achieve accurate tuning of the resonance frequency of the RF coils. According to some embodiments of the invention, overlap topology (array formations) is applied to minimize mutual coupling between coil elements. As is discussed further below, coil systems in accordance with various embodiments of the invention can be used with MRI technology to provide imaging of various regions of the body.

MRI RF coils, including both volume (such as those for whole-body imaging) and surface (such as those for localized area scanning) coils, are typically rigid, often causing patient discomfort and movement during the scanning process, leading to inaccurate imaging and increased clinical time. In addition, it may be difficult to conform the rigid coil to curved joints or other body regions. Because the rigid coils cannot be placed in close proximity to the imaged area of the body, noises are often received from other parts of the human body not intended for imaging. This results in a relatively lower SNR, and thus the RF coil serves as a limiting factor for high-performance imaging in which high resolution and detailed information about imaged features, such as but not limited to arterial plaque components, are desired.

Coil structures according to certain embodiments of the invention exhibit fabric-like flexibility, high durability for repeated flexion, and/or increased coverage area with the integration of multiple coil elements. By using electro-textiles, RF coils according to many embodiments of the invention are flexible and can conform to regions of interest to be imaged. An electro-textile may include one of a variety of types of conductive wire being flexibly fixed to one of a variety of types of textiles, as further discussed in relation to electro-textile RF coil elements below. Further, the use of ergonomic coils may reduce imaging errors due to patient motion relative to the MRI scanner resulting from discomfort and/or inability to remain immobile. This can potentially increase hospital throughput of MRI scans.

The flexibility of electro-textile RF coils in accordance with various embodiments of the invention may also lend to high durability through repeated uses. The durability of the coil, according to many embodiments of the invention, makes it practical for clinical use and reduces maintenance costs for hospitals. In addition, by integrating multiple coil elements using multiple-layer structures, coil arrays according to some embodiments of the invention provide coverage of larger areas than single element coils, allowing medical professionals to view areas of interest more extensively.

### Magnetic Resonance Imaging (MRI)

A basic description of MR physics at a general level follows. Atoms with an odd number of protons or neutrons possess a nuclear spin angular momentum, and therefore exhibit the MR phenomenon. Qualitatively, these nucleons can be visualized as spinning charged spheres that give rise to a small magnetic moment. These MR-relevant nuclei may be referred to as spins. Hydrogen with a single proton is considered to be the most abundant (as the body consists largely of H₂O), the most sensitive and the most studied. The angular frequency of the electromagnetic fields ωᵣ is given by the Larmor equation Eq. 1

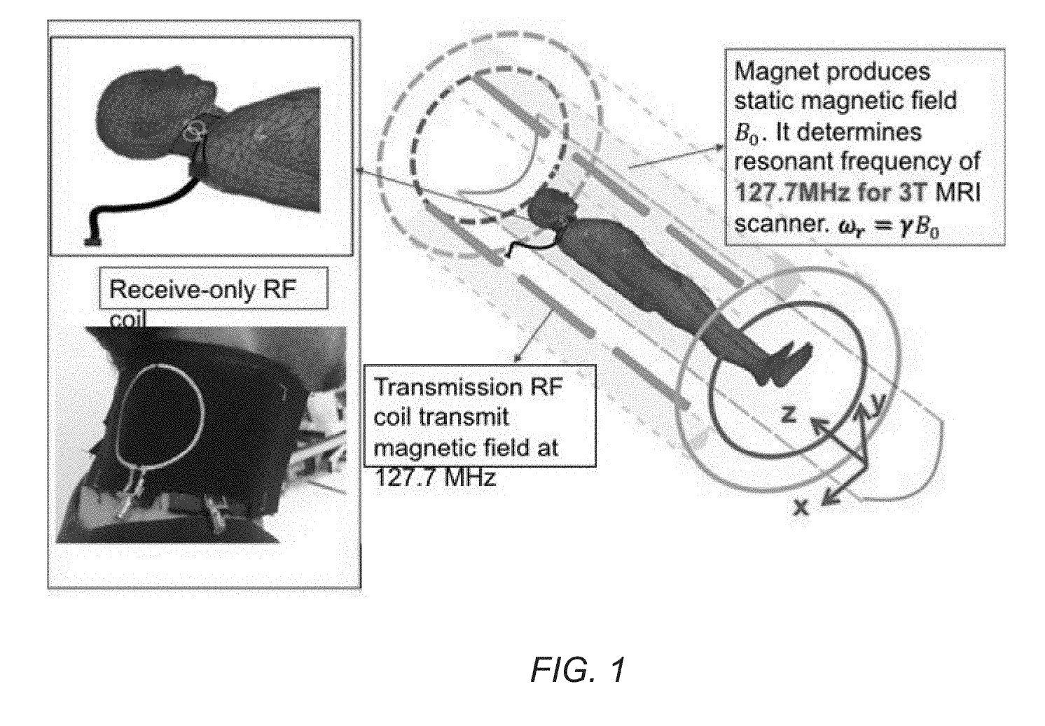

Ωᵣ=γB₀ (1)

where B₀ denotes the strength of the static magnetic field, and is known as the gyromagnetic ratio. For protons, γ/2π=42.58 MHz per Tesla. The nature of MR is based on the interaction of the spins with three types of magnetic fields: 1) main field B₀, 2) radio frequency field B₁, and 3) linear gradient fields G. An MRI scanner is shown in FIG. 1. The MRI scanner cutaway exhibits 1) the main magnet that produces field B₀ in the z direction, 2) the radio frequency coil that produces field B₁ typically in the xy plane, and 3) the gradient coils that add incremental difference in the strength of B₀ in x, y or z directions, to encode the spatial information into the signal received.

### RF Coils in MRI Scanners

RF coils are an integral part of MRI scanners, and are used for the transmission and reception of RF magnetic signals. When the imaged area is not the entire human body, surface RF coils are often preferred due to their advantages in improving SNR. They spatially reject noise from parts of the human body that are not being imaged and as a result inherently have higher SNR than whole-body volume coils. Current RF coils (such as but not limited to that shown for neck imaging in the lower left part of FIG. 1), however, typically have the disadvantage of uncomfortable wear. Given the longer imaging time required for performing an MRI scan as compared to other imaging modalities, this disadvantage may consequently lead to other problems such as patient non-compliance as to immobility, repetitive imaging trials, MRI image motion artifacts, and other issues leading to poor imaging results. Additionally, the high curvature nature of certain areas of the body, such as but not limited to the neck, shoulders, knees, elbows and ankles, can create difficulty in conforming RF coils in close proximity to the shape of the area for imaging, often resulting in reduced SNR.

Flexible RF coils can improve SNR by spatially rejecting noise from parts of the body that are not being imaged. Noises in MRI imaging can come from two different sources: human tissue and coils along with its relevant circuitry. For many clinical applications of RF surface coils, the noise from human tissue is typically greater or comparable to that from coils and circuitry. Thus, a slight increase in losses by coils and circuitry may be offset by the advantages of ergonomic considerations and potentially higher SNR of flexible RF surface coils. Flexible MRI RF coils created using screen printing, ink-jet printing and copper braid have been studied. The substrates of the first two methods have been shown to exhibit limited flexibility as compared to typical cloth material. Additionally, the second method can be limited by the thickness of the conductor that can be printed. As a result, its conductivity may be limited at lower frequencies. The third method uses meandered copper braid and as a result may be worn out over time.

In many embodiments of the invention, electro-textiles are utilized to form a multi-layer flexible RF coil array system for use in MRI imaging, such as but not limited to 3T MRI carotid artery imaging. Electro-textiles often have the advantages of being highly conductive, flexible like fabric and long lasting. In certain embodiments of the invention, the RF coil system operates at 127.7 MHz, as determined by the static magnetic field of a 3T MRI system. As can readily be appreciated, the RF coil system can be implemented to achieve a resonant frequency appropriate to the requirements of a given imaging application. In some embodiments of the invention, RF coil elements are optimized at a required resonant frequency and input impedance matched to 50Ω, with and without incorporating active tuning.

While various MRI RF coils are described above with respect to FIG. 1, other coils may be utilized as appropriate to the requirements of a specific application in accordance with various embodiments of the invention. Electro-textile RF coil elements in accordance with a number of embodiments of the invention are discussed further below.

### Electro-Textile RF Coil Elements

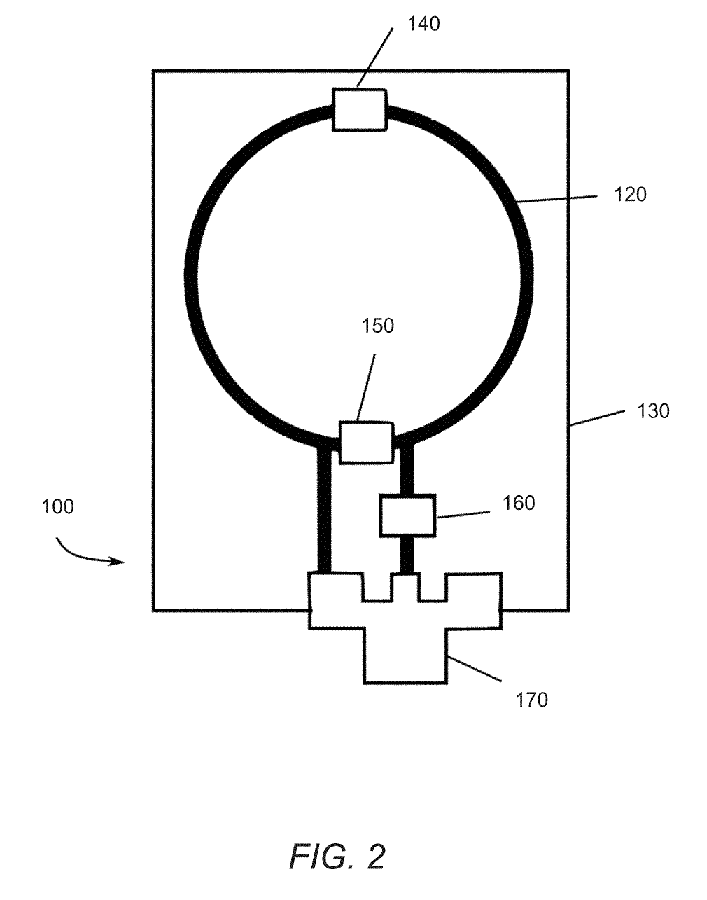

FIG. 2 illustrates a front view of a single electro-textile RF coil element 100 according to an embodiment of the invention. In some embodiments of the invention, a single element electro-textile coil 100 includes the coil pattern 120. The coil pattern 120 may be embroidered into, adhered onto, or otherwise flexibly fixed onto a fabric 130. Fabric 130 may be formed using one of various types of textiles, cloth, or other flexible, porous and/or insulating material. In many embodiments of the invention, fabric 130 is a flexible material including a network of natural or artificial fibers of, for example, yarn or thread. The fiber may be produced from, for example, animal, plant, mineral, and/or synthetic sources. Fabric 130 may be created using one or more of a variety of methods, including but not limited to weaving, knitting, crocheting, knotting, or felting. Weaving may involve two distinct sets of yarn or thread being interlaced over and under each other. Knitting and crocheting involve interlacing loops together in a line. Knotting involves fastening or securing rope, yarn or thread by tying or interweaving. Felting includes textile production by matting, condensing, and pressing fibers together. In addition, fabric 130 may be created by thermal, mechanical and/or adhesive bonding of fibers.

Fabric 130 may exhibit one of various strengths, thicknesses, flexibility, durability, elasticity and other properties. It may have flexibility sufficient to conform closely to certain human body structures, such that at least a majority or entirety of the coil pattern 120 is in contact with a certain body structure. It may have sufficient elasticity such that it remains secured to a region of interest to be imaged, despite movement of the patient. Additionally or alternatively, it may have limited elasticity to, for example, aid in preserving the shape of the coil pattern 120.

In many embodiments of the invention, coil pattern 120 may be formed using one or more of various types of electro-textile materials, such as conductive textile thread or fiber, or any of a variety of conductive, flexible materials. Fiber may include a fine, slender filament, or a natural or synthetic substance that is significantly longer than it is wide. Thread may include a fine cord or other fibrous material spun out to considerable length, and may be composed of two or more filaments twisted together. The fiber or thread forming coil pattern 120 may be one or more of a variety of materials capable of conducting RF signals, such as but not limited to conductive metal threads (silver, copper, aluminum, brass, etc.), carbon based conductive materials, metal plated on natural or synthetic chord, and conductive materials mixed with non-conductive threads.

In many embodiments of the invention, the coil pattern 120 may be embroidered or otherwise flexibly fixed to fabric 130, using one or more of a variety methods, such as stitching, sewing, knitting, weaving, or other form of threading conductive fiber or thread through or between crossing or adjacent fibers of fabric 130. As one example and not by way of limitation, conductive thread may be stitched, or threaded back and forth between the front and back sides of fabric 130, to form coil pattern 120.

In other embodiments of the invention, coil pattern 120 may be formed using conductive textile cloth. The conductive cloth may include a pliable material made using one of various techniques such as but not limited to weaving, felting, or knitting natural or synthetic fibers, filaments or threads. The fibers, filaments and/or threads forming the conductive cloth may include one or more of a variety of materials capable of conducting RF signals, such as but not limited to conductive metal threads (silver, copper, aluminum, brass, etc.), carbon based conductive materials, metal plated on natural or synthetic chord, and conductive materials mixed with non-conductive threads.

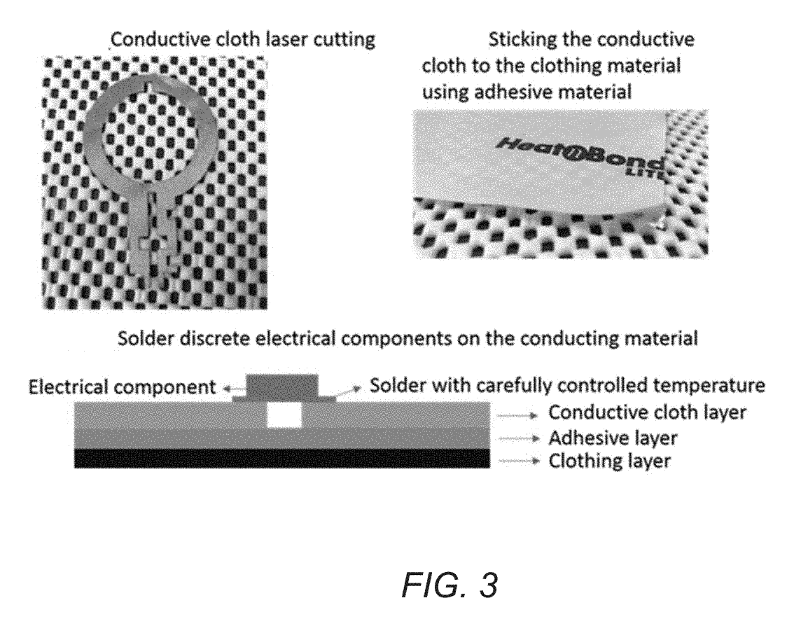

In some embodiments of the invention, accurate cutting technologies, such as but not limited to laser cutting, waterjet cutting, and plasma cutting, can be used to cut the conductive cloth into the pattern needed. An example of a cut pattern according to certain embodiments of the invention is shown in FIG. 3.

According to some embodiments of the invention, in constructing the coil pattern 120 using conductive cloth, one or more tracing templates may be employed to cut the conductive cloth according to an accurate outline. In some embodiments of the invention, the tracing template may include only the desired outline of the coil pattern 120. In some cases, the desired coil pattern 120 is formed using a single, connected outline. In other cases, the tracing template may provide for space between portions of the coil pattern 120 to accommodate structures such as, but not limited to, tuning components. Alternatively, the tracing template may outline the desired coil pattern as well as areas extraneous to the desired coil pattern 120. The extraneous portions may be subsequently removed by cutting or other methods, either before or after being fixed to fabric 130 as described below.

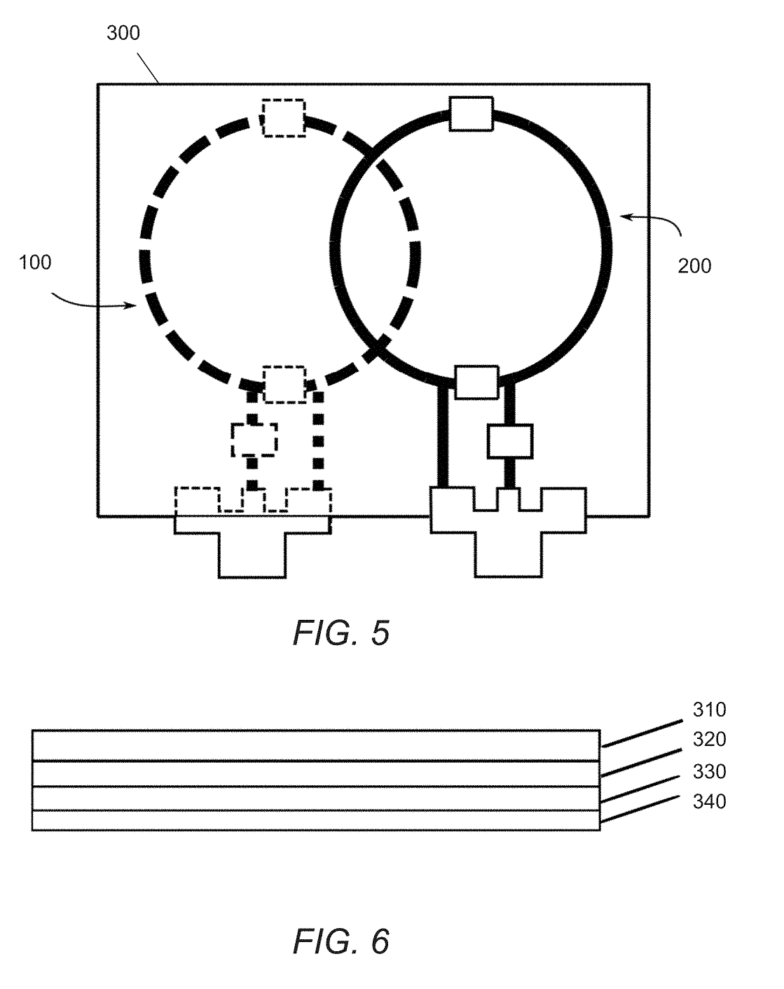

When forming coil arrays such as that described below in relation to FIGS. 5 and 6, the coil patterns of the multiple elements may be cut individually and then aligned as appropriate to form the array. Alternatively, connecting portions between multiple elements may be outlined, cut and subsequently removed.

In many embodiments of the invention, the cut conductive cloth forming coil pattern 120 can be flexibly fixed to fabric 130 using an adhesive material other appropriate method, such as but not limited to stitching. In many embodiments of the invention, the dielectric loss requirement for the adhesive material is not very rigorous, allowing for low cost manufacture and scalability. Various types of adhesives can be used, including but not limited to heat-activated, sewable, double-sided, spray-on, permanent, or other types of fabric or multi-purpose adhesives. The adhesive material can be, for example, a commercially available adhesive such as but not limited to HeatnBond Lite by Therm O Web of Wheeling, Ill., as shown in FIG. 3. The adhesive material may also be pre-affixed to a surface of the conductive cloth.

In certain embodiments of the invention, discrete electrical components such as but not limited to capacitors, inductors and diodes are soldered onto the conductive cloth using, for example, industrial standard solder, as indicated in FIG. 3. The soldering process may be performed under carefully controlled temperatures and procedures, such as limiting the amount of contact time between the touch point of the solder and the conductive cloth. As one example, for purposes of illustration and not by way of limitation, the soldering temperature may be set at 400 degrees Celsius. This temperature can be sufficiently high to melt solder such as, for example, Loctite multicore wire solder by Henkel AG & Company, KGaA, headquartered in Düsseldorf, Germany, while avoiding damage to certain types of conductive cloth. Limiting the direct contact between the high-temperature solder and conductive cloth to, for example, 5 seconds or less may prevent damage to the conductive cloth.

In a number of embodiments of the invention, the soldered discrete electrical components experience lower dielectric loss than, for example, capacitors used with other technologies such as but not limited to screen printing, which have limitations as to the values of the capacitance and inductance that can be achieved. The use of cut conductive cloth also allows a variety of discrete electrical components with appropriate properties to be integrated with coil pattern 120, in comparison to other technologies such as (but not limited to) screen printing that can lead to great difficulties in integrating more complicated electrical components.

According to some embodiments of the invention, fabric 130 and/or coil pattern 120 may be flexed over 90 degrees without changing electrical properties of coil 100. Fabric 130 and/or coil pattern 120 may be bent with forces in different directions, to form complex shapes and curvatures with concave and incurvate shapes. In many embodiments of the invention, fabric 130 and/or coil pattern 120 may be sufficiently stable, such that other components may remain durably fixed, and the original performance of the coil may be preserved, after numerous repeated instances of flexion.

Coil pattern 120 may be formed as a circle, hexagon, rectangle or any other shape appropriate to the requirements of a given application, and may be shaped with gaps and/or branches. According to certain embodiments of the invention, the use of a circular shape facilitates fabrication using embroidery machines, as it may be less susceptible to broken or trapped threads, given the relatively evenly distributed needle movements in creating a circular shape.

In certain embodiments of the invention, coil 100 may include tuning components 140, 150 and 160 such as but not limited to capacitors, varactors or variable capacitors used for frequency tuning and impedance matching of the RF coil. The tuning components may connect gaps within the coil pattern 120, and can be used to accurately tune a resonance frequency to a designated frequency, depending on the specific application. In some embodiments of the invention, an RF connector 170, such as but not limited to a SubMiniature version A (SMA) connector, may be coupled with branches of coil pattern 120. In other embodiments of the invention, RF cables may be attached directly to coil pattern 120 using one of various methods such as but not limited to soldering.

While various electro-textile RF coil elements are described above with respect to FIGS. 2-3 and 5-6, other coil elements may be utilized appropriate to the requirements of a specific application in accordance with various embodiments of the invention. Conductive textile threads in accordance with a number of embodiments of the invention are discussed further below.

### Conductive Textile Threads

In some embodiments of the invention, conductive textile threads used to form coil patterns in flexible RF coils are metal-coated textiles chosen for their balance of conductivity, tensile strength, diameter and flexibility. The conductive threads can, for example, be anti-static, anti-bacterial, silver- or other metal-plated, made of polyamide or another synthetic material, monofilament, multifilament, twisted, incorporated with non-conductive thread, tear-resistant, anti-tarnish, have high electrical conductivity, and/or have high thermal conductivity. The conductive threads can also, for example, be formed of high-performance multifilament yarn spun from liquid crystal polymer (LCP), be 72% lighter than 30 AWG copper wire, have a break strength 5 times greater than 30 AWG (stranded 40/46) copper wire, feature very low thermal expansion, and/or surpass much heavier copper-based braids in shielding performance.

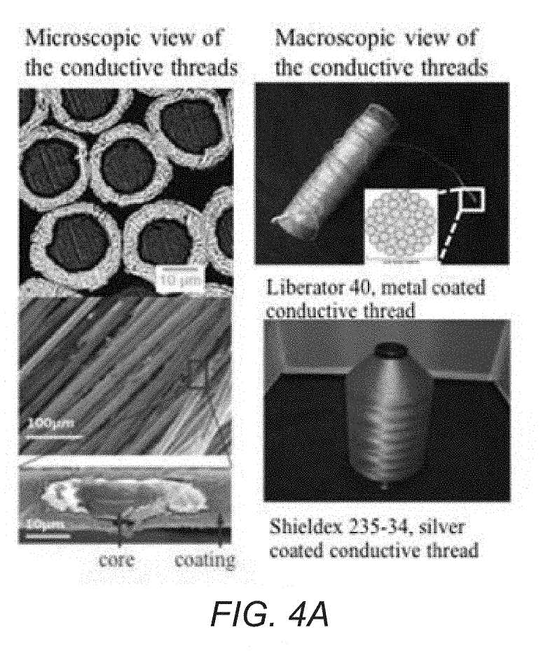

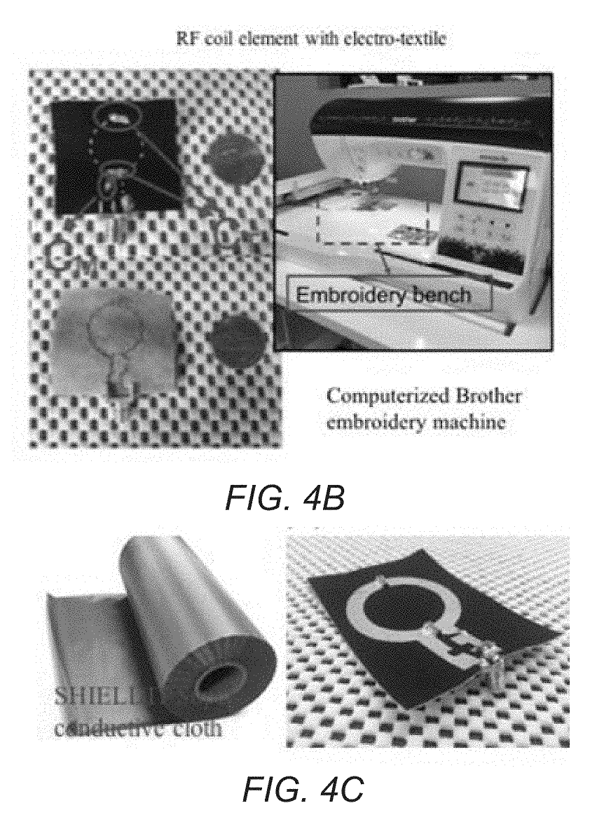

As examples and not by way of limitation, FIG. 4A shows microscopic and macroscopic views of illustrative electro-textile threads that can be employed in certain embodiments of the invention: the Shieldex 235/34 by Shieldex-U.S. of Palmyra, N.Y., and Liberator 40 by Syscom Advanced Materials of Columbus, Ohio Various other types of electro-textile threads that may be employed, as examples and not by way of limitation, are the Shieldex 110/34 dtex 2-ply HC by Shieldex-U.S., the Agsis 100D, Agsis 200D, Liberator 20, and Amberstrand Lyofill 166 of Syscom Advanced Materials, and thin copper-based electro-textiles such as those produced by Elektrisola Feindraht AG of Switzerland.

The Shieldex 110/34 dtex 2-ply HC thread (hereinafter the “Shieldex thread”) is created from two strands of raw yarn. It has a diameter of 0.2 mm, similar to standard cotton-based threads used for embroidery machines. With a nylon core, the flexibility of the Shieldex thread is similar to that of standard nylon-based threads, making it suitable for embroidery machine integration. The Shieldex thread has a high and varied DC linear resistivity of 500 Ωm±100 Ω/m.

The Liberator 40 is made of 40 filaments of Kururay Vectran fiber coated with 3 μm silver or copper layers. Liberator 40's DC linear resistivity is 3 Ω/m with an approximate diameter of 0.5 mm. The polymer-based Vectran fiber has a tensile strength of 3 GPa, which is more than four times the strength of nylon. Although it is less flexible than the Shieldex thread, its high tensile strength makes it less vulnerable breakage during embroidery.

While various types of conductive textile threads are described above with respect to FIG. 4A, other conductive threads may be utilized appropriate to the requirements of a specific application in accordance with various embodiments of the invention. Conductive electro-textile cloth in accordance with a number of embodiments of the invention are discussed further below.

### Conductive Electro-Textile Cloth

In a number of embodiments of the invention, conductive electro-textile cloth, such as but not limited to that shown in FIG. 4C, is used to form the coil pattern in the RF coil. Using accurate cutting technologies, such as but not limited to laser cutting, the conductive coil pattern can be formed. In some embodiments of the invention, the resistivity of the conductive cloth is between 0.05 Ω/sq and 1 Ω/sq. One example of a fabricated RF coil element, using the electro-textile cloth SHIELDIt Super by Less EMF Inc. of Latham, N.Y., is shown on the right of FIG. 4C. Various other types of conductive electro-textile cloth that may be used are, for example and not by way of limitation, CobalTex, Copper RipStop Fabric, Pure Copper Polyester Taffeta Fabric, RipStop Silver Fabric, and Stick E Shield by Less EMF Inc.

In some embodiments of the invention, the conductivity of the conductive cloth can be 10⁶ S/m or higher, given a frequency of 120 MHz. In certain embodiments of the invention, the conductivity of the conductive cloth can be between 10⁵ S/m and 10⁶ S/m. The conductive cloth, according to certain embodiments of the invention, may be formed with conductive metals that are non-magnetic, and/or may exhibit a smooth surface, which may allow for uniform conductivity in various directions. In many embodiments of the invention, the conductive cloth used is resistant to changes in electrical and mechanical properties at high temperatures, such as but not limited to, 200 degrees Celsius or higher. The conductive cloth used in many embodiments of the invention exhibits a tensile strength such that the coil pattern created using the conductive cloth can withstand repeated use and flexion.

In certain embodiments of the invention, the conductive cloth used to form coil pattern 120 can include shielding fabric capable of radiofrequency and microwave shielding, can be made of polyester substrate and/or other tear-resistant material, and/or can be plated with nickel, copper, or other conductive substance. The resistivity of a surface of the conductive cloth may be, but is not required to be, <0.5 Ohm/sq.

While various types of conductive electro-textile cloth are described above with respect to FIG. 4C, other types of conductive cloth may be utilized appropriate to the requirements of a specific application in accordance with various embodiments of the invention. RF coil arrays in accordance with a number of embodiments of the invention are discussed further below.

### RF Coil Arrays

FIG. 5 illustrates a front view of a two-element multi-layer electro-textile RF coil array 300 according to an embodiment of the invention. According to many embodiments of the invention, a multi-element electro-textile RF coil array 300 can include multiple RF coil elements, such as but not limited to elements 100 and 200 shown in FIG. 5. Elements 100 and 200 may each function as an independent coil such as that described in relation to FIG. 2, and may each be constructed using a variety of materials and methods such as that described in relation to FIG. 2.

The coil array 300 according to many embodiments of the invention is implemented using a multi-layer structure including but not limited to, for example, layers 310/320/330/340 shown in FIG. 6. As an example but not by way of limitation, layer 310 may include element 100 of FIG. 5, and layer 330 may include element 200 of FIG. 5. Layer 320 may include fabric or other flexible insulating material to separate the conducting components of layers 310 and 330. Layer 340 may include fabric or other flexible insulating material to provide separation between the coil array structure 300 and a patient's body. The material used in layers 320 and/or 340 may include fabric formed using one of various types of textiles, cloth, or other flexible, porous and/or insulating material, such as but not limited to that described in the above section in relation to fabric 130 of coil element 100.

The multi-layer structure 300 can be formed using a variety of methods. According to a number of embodiments of the invention, the layers may be stacked, with adhesive materials being used outside the coil patterns to bond the layers together. Additionally or alternatively, the layers may be stitched together using non-conductive thread. In some embodiments of the invention, one side or section of the coil array 300 may be fixed together using one or more of a variety of attachment methods, and fastener may be used on another side or section to accommodate for different flexion angles of the coil array. The fastener may include one or more of a variety of structures for temporarily or permanently securing layers of the coil array together, such as but not limited to a clip, screw, pin, string, wire, chain, hinge, button, clasp, hook-and-eye fastener, hook-and-loop fastener, buckle, strap, elastic, or any other appropriate fastener.

In some embodiments of the invention, coil patterns of the coil elements are overlapped but disposed so as to not directly come in contact with each other so as to, for example, reduce mutual coupling between the elements. The amount of overlap between elements may be determined based on optimal imaging performance, and/or lowest coupling. As an example and not by way of limitation, in order to achieve low mutual interaction between coil elements, an overlap distance of approximately 0.48 of the radius of circular coil patterns may be used in certain applications. In some embodiments of the invention, the overlap distance is measured by the distance between the outermost points of each circle, along a line connecting the center of the two circles. The overlap distance may be calculated based on conditions in free space. In many embodiments of the invention, the overlap distance may be determined so as to minimize coupling between the two coil elements, and thus maximize SNR of the coil array.

Various factors may affect the amount of overlap distance that minimizes coupling, including but not limited to the body region being imaged, type(s) of conductive material used in the coil patterns, and the type(s) of fabric used in the various layers. In certain embodiments of the invention, the overlap distance may be determined through detailed electromagnetic full wave analyses. This may be performed by computing the coupling coefficient among the two or more coils by varying the separation (i.e., overlap distance) of the coils, for a given geometrical configuration of coil patterns, type of material used between the coil patterns, and type of conductive material used to form the coil patterns. Results of the overlap distance may be subsequently verified through measurements.

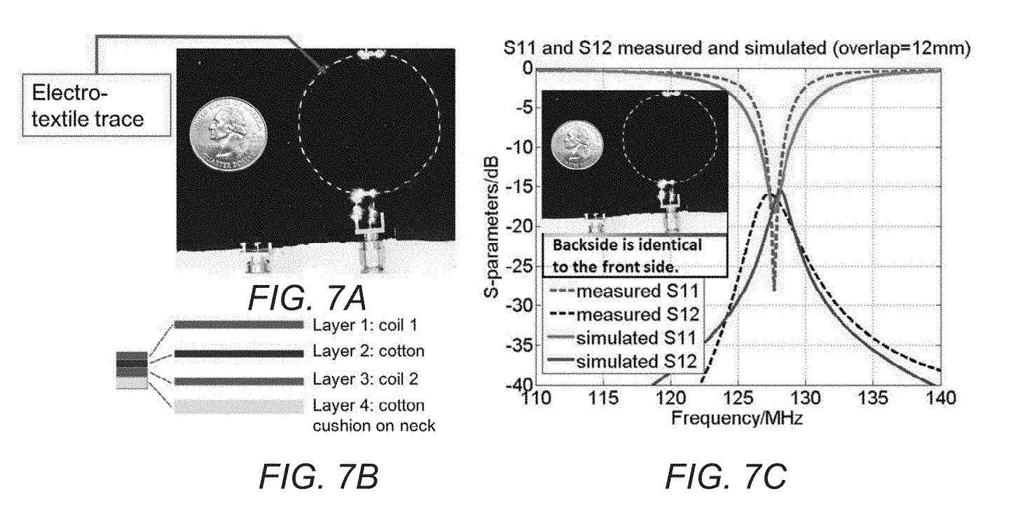

An example of a coil array structure according to some embodiments of the invention is illustrated in FIG. 7B. FIG. 7A shows a front view of a fabricated coil array with electro-textiles, with one coil element in front and the other in back; and FIG. 7B a side view of a 4-layer coil array structure with electro-textiles; according to many embodiments of the invention.

According to certain embodiments of the invention, the multi-layer structure of the RF coil array enables increased coverage area while maintaining effective imaging performance. Usage of embroidered coil patterns or conductive cloth according to some embodiments of the invention allows for a high level of flexibility and durability. The number of coil elements used, and the specific arrangement thereof, can vary depending on the shape and size of the region targeted for imaging.

While electro-textile RF coil arrays are described above with respect to FIGS. 2, 5-6 and 7A-B, other coil arrays may be utilized appropriate to the requirements of a specific application in accordance with various embodiments of the invention. Flexible coil cases in accordance with a number of embodiments of the invention are discussed further below.

### Flexible Coil Case

According to some embodiments of the invention, RF coils are enclosed in a case configured to fit around and/or conform to designated regions of the body for imaging. The case can be constructed using fabric, elastic, and/or any of a variety of other flexible and/or stretchable materials. The structure of the case can be shaped to conform to a localized anatomical region, such as but not limited to the neck, knee, ankle, elbow, wrist, shoulder, or any other part of the body. The coil case can be fastened to and/or around the region for imaging using one or more of a variety of methods, including but not limited to a sticky pad, clip, screw, pin, string, wire, chain, hinge, button, clasp, hook-and-eye fastener, hook-and-loop fastener, buckle, strap, elastic, and/or any other appropriate fastener.

According to certain embodiments of the invention, the coil case includes one or more openings to allow the RF coil to be inserted and removed. This may allow for ease of repair and update of the coil. In several embodiments of the invention, a fastening mechanism is included at one or more portions of the coil case, such that a disposable layer of material, such as but not limited to medical grade non-woven fabrics, can be temporarily fastened. The disposable layer may provide a hygienic separation between the surface of a patient's body and the coil case.

Coil cases in a number of embodiments of the invention include one or more mechanisms on the interior and/or exterior of the case to prevent cables from being tangled or wound together. As an example and not by way of limitation, a grating structure may be used. One or more openings in the case may also be included to allow for cables to be hidden inside the case and exit from the opening(s). In certain embodiments of the invention, thin and flexible RF cables are used to, for example, maintain the flexibility of the coil. In some embodiments of the invention, RF connectors may be removed to, for example, preserve flexibility, reduce weight and the cost of the RF coil, and the RF cables attached directly to the coil pattern.

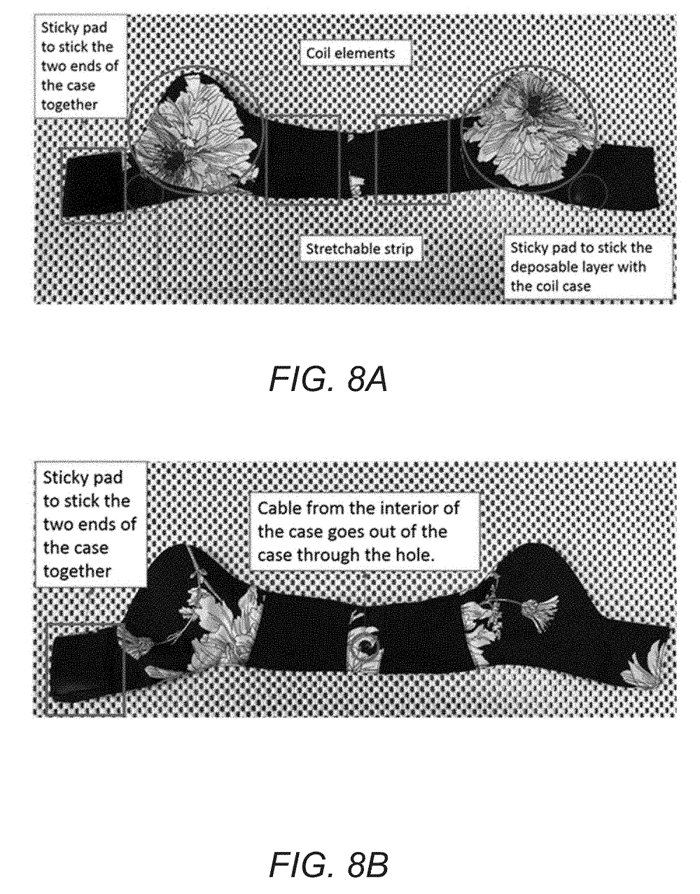

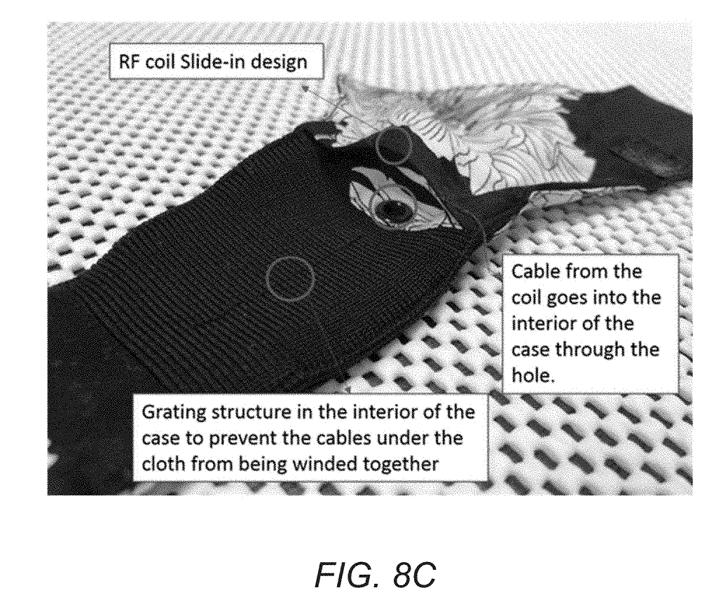

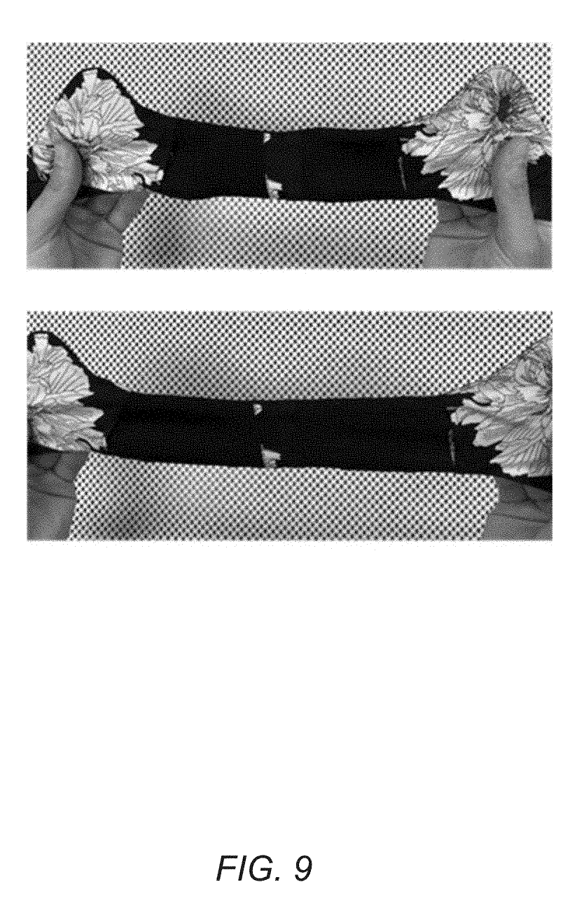

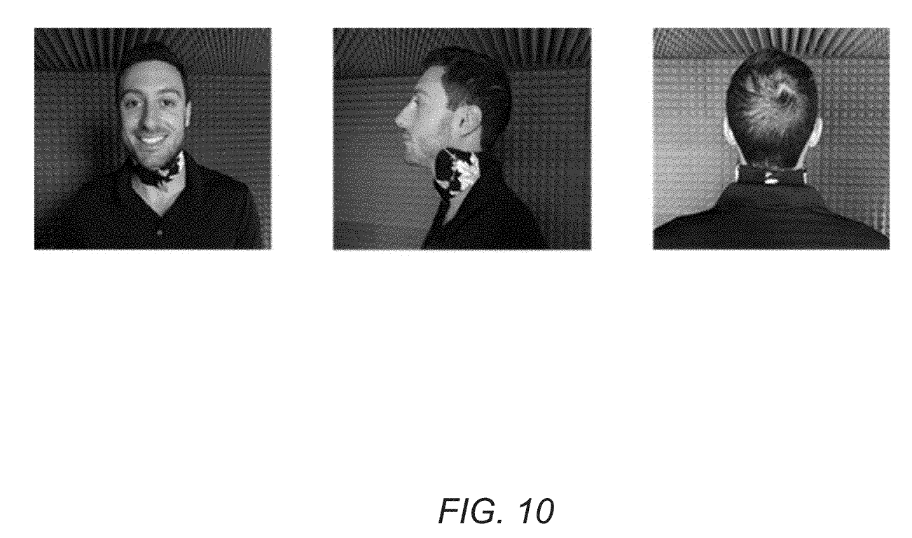

One example of a coil case according to some embodiments of the invention is shown in FIGS. 8A-8C, 9 and 10. This particular coil case is designed to be fastened around a patient's neck by using sticky pads and stretchable material. The case is configured to conform to the complex geometry of the neck by placing and/or fastening together different sections of the case to create the correct curvature.

While flexible coil cases are described above with respect to FIGS. 8A-8C, 9 and 10, other coil cases may be utilized appropriate to the requirements of a specific application in accordance with various embodiments of the invention. MRI RF coil element design in accordance with a number of embodiments of the invention are discussed further below.

### MRI RF Coil Element Design

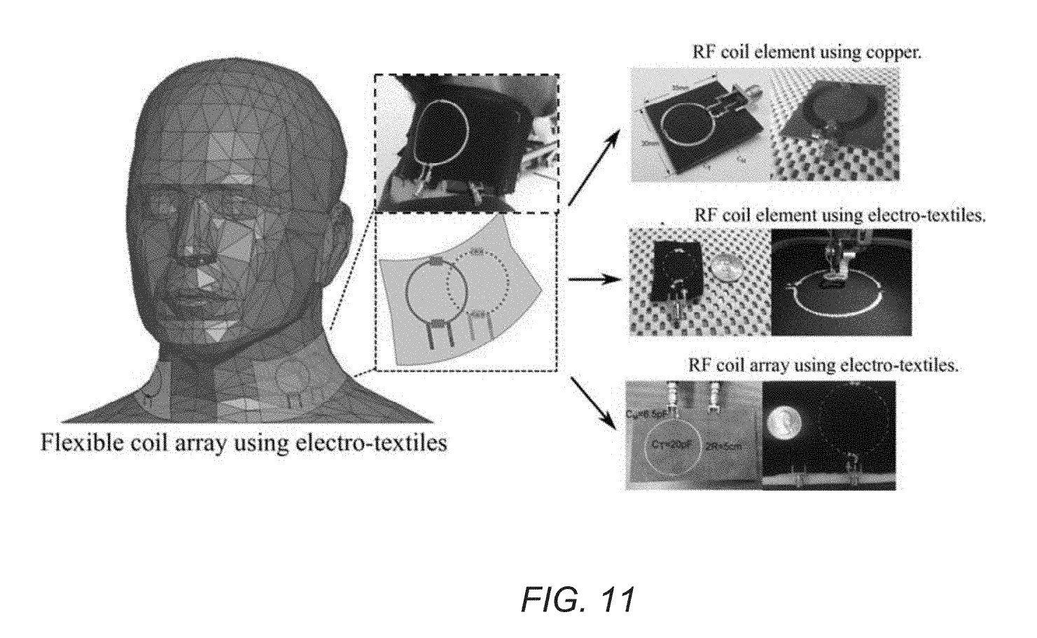

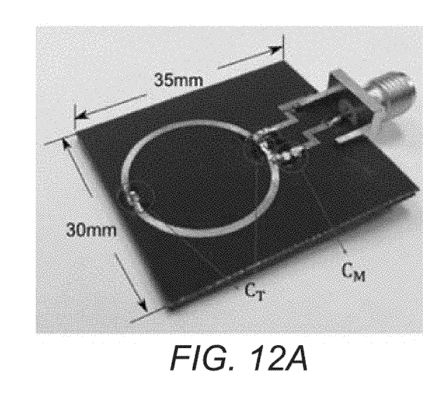

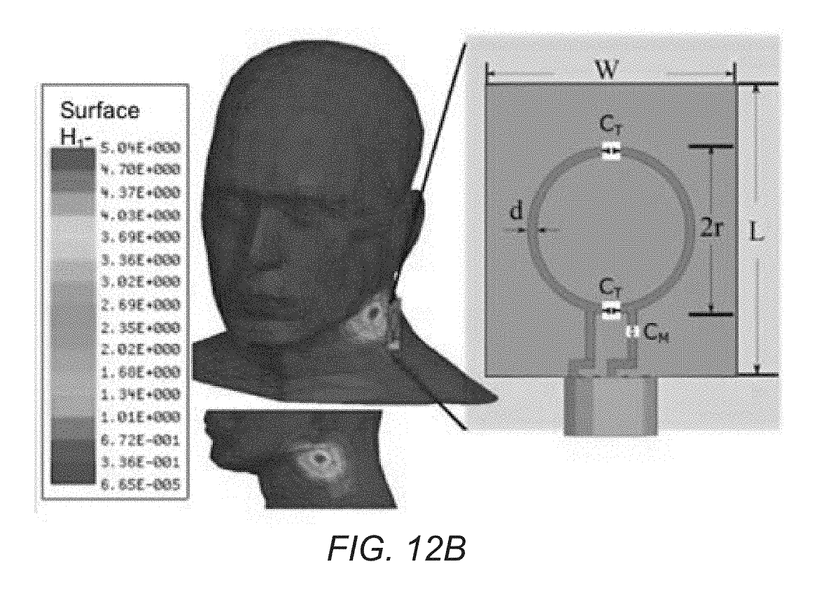

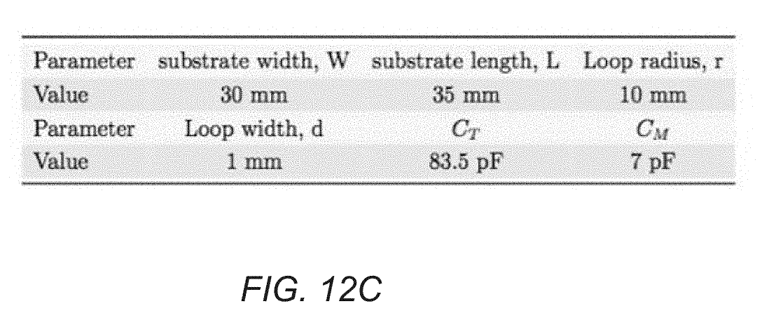

RF coils in many embodiments of the invention are designed for use with 3T MRI systems at a frequency of 127.74 MHz. The RF coils can be used in transmit/receive coil systems, can be used as a receiving only coil, or a coil array, such as in the examples shown in FIG. 11. The structure of an RF coil according to some embodiments of the invention, for use at a position relative to the human neck, are shown in FIGS. 12A and 12B. FIG. 12C lists parameter values of an example RF coil according to certain embodiments the invention. The RF coil may be placed a short distance, such as but not limited to 10 mm, away from the patient's neck. In some embodiments of the invention, the coil is connected to an RF connector, such as but not limited to an edge mount 50Ω SMA connector. The example coil shown in FIG. 12A is fabricated with photolithography on FR4 glass-reinforced epoxy laminate substrate, with specifications of r=4.4, tan=0.02 and a thickness of 0.4 mm. In many embodiments of the invention, no balun is included because simulations show that the current on the outer side of the coaxial cable is negligible.

In designing coil elements and arrays for use with MRI systems, different parameters can be adjusted to achieve resonant tuning and impedance matching. As an example, coils for use with 3 Tesla MRI machines should be tuned to a resonance frequency of 127.7 MHz. This can be a challenging process when using electro-textiles due to the narrow bandwidth of the coil and the RF transmission signal. The conventional methodology for tuning conventional rigid coils can involve a lengthy trial and error process near a human phantom. As discussed in the section below regarding coil array performance, appropriate RF coils according to certain embodiments of the invention are achievable without tunable circuitry.

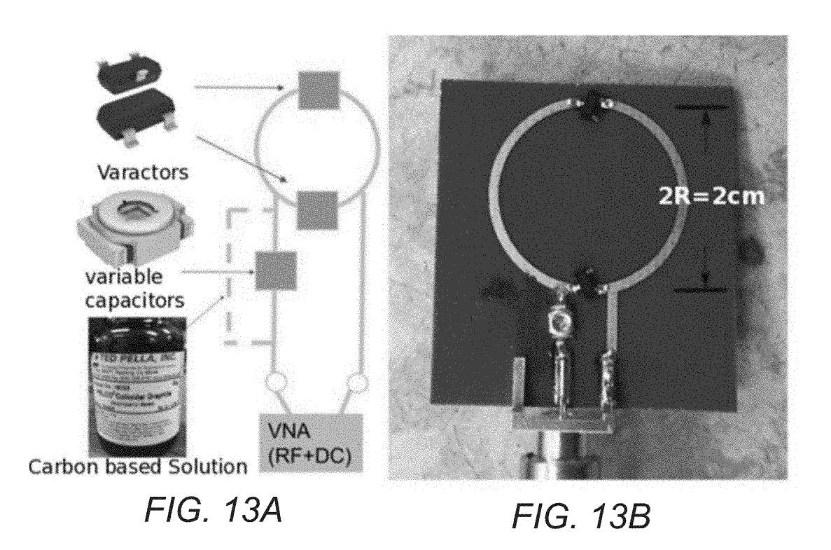

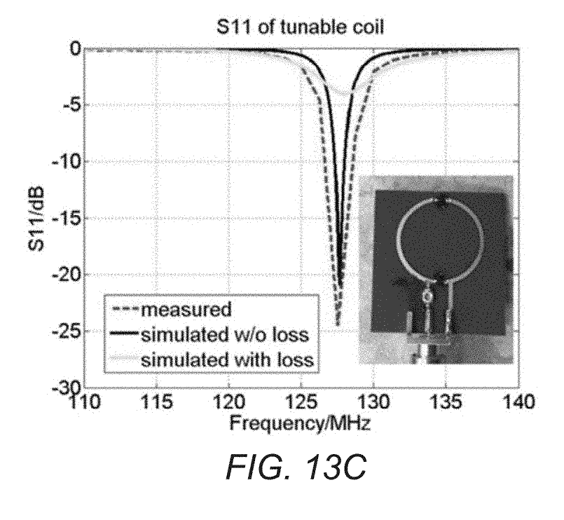

Alternatively, according to many embodiments of the invention, tunable circuitry with, for example, varactors and/or variable capacitors, can accurately tune the resonant frequency to a target frequency. An example is shown in FIG. 13A of components and design of a coil element capable of being adjusted to an accurate resonance frequency; and in FIG. 13B of a fabricated coil element with tuning circuitry. FIG. 13C shows the measured S₁₁ of this particular example of a fabricated tunable RF coil element being −24.5 dB at exactly 127.7 MHz. It can be seen that the simulation results are consistent with the measured results. Additionally, when the loss of the capacitors are not included, the impedance matching is degraded In certain embodiments of the invention. PELCO opropanol-graphite-based paint is used for DC bias. The series resistance associated with the varactors can also be considered using, for example, full wave simulation for accurate modeling.

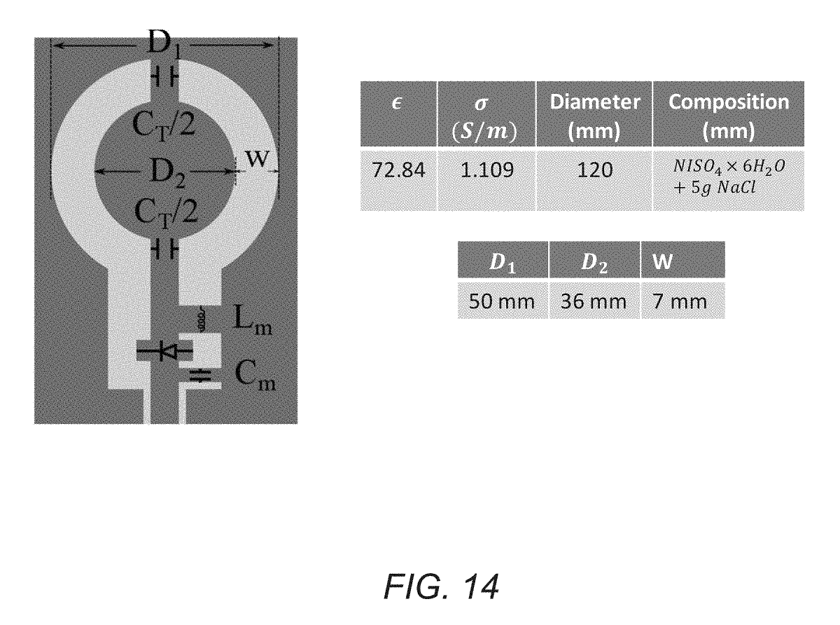

In some embodiments of the invention, the resonant frequency of 127.7 MHz and matching are achieved by using appropriate coil dimensions and capacitor loading. High SNR can be achieved, for example, by choosing a loop radius such that the magnetic field at the target depth is strongest. FIG. 14 shows a diagram of an RF coil according to certain embodiments of the invention, and its specifications, where C_{T}/2 indicates the capacitance values of the two capacitors used for frequency tuning; Cₘ indicates the capacitance value of the impedance matching capacitor; Lₘ indicates the inductance value of the inductor used for transmission decoupling; D₁ and D₂ indicate the outer and inner diameters of the coil element, respectively; and W indicates the thickness of the coil element. In some embodiments of the invention, a diode is used together with the inductor and capacitors to effect the transmission decoupling.

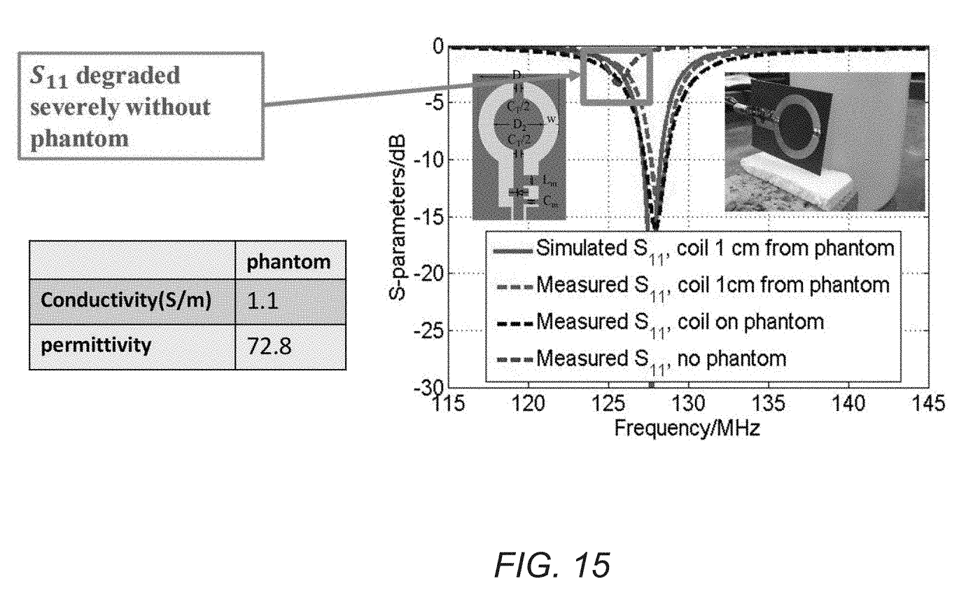

In order to accurately tune an RF coil according to some embodiments of the invention, the presence of an appropriate phantom may be required. For example, tissues in the human neck around 127 MHz are lossy and may shift resonant frequency and degrade the impedance matching. As the RF bandwidth for MRI applications is usually on the order of tens of KHz, proper coil design can depend on the presence of a phantom. As shown in FIG. 15, the S₁₁ with the presence of a phantom is −20 dB at the exact resonant frequency of 127.7 MHz. The S₁₁ without a phantom however, is shifted by 2 MHz with minimum of −4 dB.

While MRI RF coil element design is described above with respect to FIGS. 11, 12A-12C, 13A-13C, 14 and 15, other design considerations may be utilized appropriate to the requirements of a specific application in accordance with various embodiments of the invention. Performance characterization of electro-textiles in accordance with a number of embodiments of the invention are discussed further below.

### Performance Characterization of Electro-Textiles

Researchers performed validation experiments in order to accurately characterize and predict the performance of electro-textiles using software simulation tools. A method was used to obtain the effective conductivity of an electro-textile pattern according to certain embodiments of the invention, and verify that the use of a surface roughness factor is still effective around 127.7 MHz range. This method was based on Z. Wang, “Electronic Textile Antennas and Radio Frequency Circuits for Body-Worn Applications,” Ph.D. Thesis, 2014 (hereinafter “Z. Wang 2014”), the relevant disclosure of which is hereby incorporated by reference.

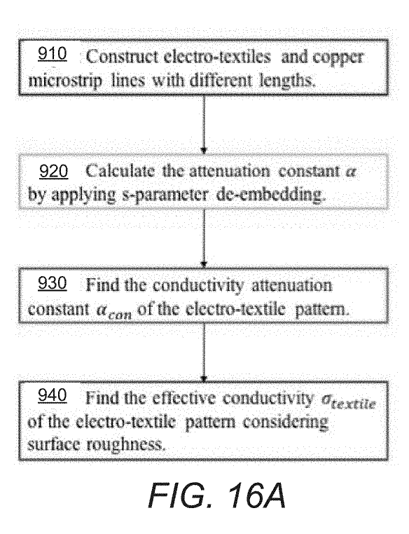

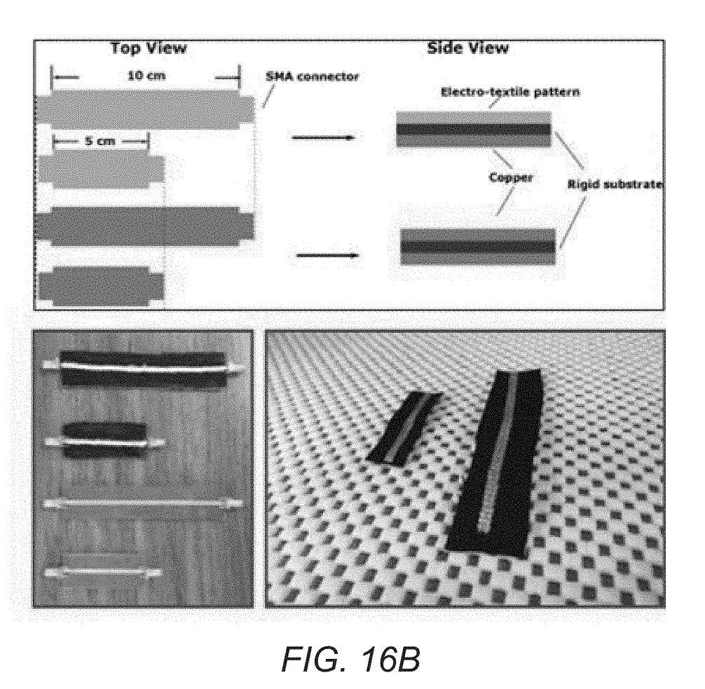

The method according to certain embodiments of the invention is illustrated in FIG. 16A. Microstrip lines of varying lengths are fabricated (910) with copper and with electro-textiles as shown in FIG. 16B. The total attenuation constant of these microstrip lines are calculated (920) based on the measured S₂₁ magnitude. The S₂₁ information may be de-embedded from the loss and perturbation of the connectors using S-matrix methods in Z. Wang 2014, and R. E. Collin, “Foundations for microwave engineering,” 2007, the relevant disclosure of which is hereby incorporated by reference. The conductivity attenuation constant of the electro-textile patterns is separated (930) from the dielectric and the radiation losses, and the effective conductivity is extracted (940) considering the surface roughness. The effective conductive loss that accounts for the loss due to surface roughness is used. The surface roughness constant K_{roughness} is used in Eq. 2 and Eq. 3 (from S. P. Morgan Jr., “Effect of Surface Roughness on Eddy Current Losses at Microwave Frequencies,” Journal of applied physics, vol. 20, no. 4, pp. 352-362, April 1949; and M. V. Lukic and D. S. Filipovic, “Modeling of 3-D Surface Roughness Effects With Application to u-Coaxial Lines,” IEEE Transactions on Microwave Theory and Techniques, vol. 55, no. 3, pp. 518-525, 2007, the relevant disclosures of which are hereby incorporated by reference) to relate the effective attenuation constant and the ordinary attenuation constant derived based on a smooth metallic surface.

α_{cond-eff}=α_{cond}×K_{roughness} (2)

where α_{cond-eff} is the effective conductivity, α_{cond} is the ordinary conductivity, and K_{roughness} is the surface roughness constant. The surface roughness constant is calculated in Eq. 3:

$${K}_{roughness} = 1 + \frac{2}{π} {tan}^{- 1} ( \frac{1.4 {Δ}_{surface roughness}}{{δ}_{skin depth}} ) \qquad ( 3 )$$

where Δ_{surface roughness} is the height of the rough surface triangle from the bottom to the top.

It was observed that when the surface roughness is greater than 6, the K factor begins to be constant at the value of K_{roughness}=2. In Z. Wang 2014, the surface roughness constant K_{roughness}=2 is used considering the large surface roughness compared with the skin depth. At a frequency of 127.7 MHz, the skin depth of the copper is around 6 m, and in order for the K factor to be 2, the surface roughness needs to be greater than 36 m. As a result, the value of surface roughness constant needs to be verified by comparing the measured S₁₁ performance of the RF coil with simulation results. The S₂₁ of the 5 cm and 10 cm electro-textile patterns around 127.7 MHz are −0.05 dB and −0.15 dB respectively. The effective conductivity is predicted as 4×10⁶ S/m, one order of magnitude lower than the copper conductivity, using the calculation method discussed above. It is observed that the loss is small compared with performance in a higher frequency range as shown in Z. Wang 2014. The vector network analyzer (VNA) error is offset by the S-matrix de-embedding process after comparing the results of multiple trials. It is verified as discussed below in the simulation of a single-thread test coil that the S₁₁ performance is accurately predicted. As a result, the use of a K factor of 2 is appropriate. In summary, the applied method to extract the effective conductivity of the pattern, according to many embodiments of the invention, is effective in predicting the performance of the electro-textile RF coils around 127 MHz.

While performance characterization of electro-textiles is described above with respect to FIGS. 16A-16B, other characterization methods may be utilized appropriate to the requirements of a specific application in accordance with various embodiments of the invention. Performance simulation of single-thread test coils in accordance with a number of embodiments of the invention are discussed further below.

### Performance Simulation of Single-Thread Test Coil

In preliminary testing, researchers constructed electro-textile RF coils using a single-thread pattern. For cases of single-thread characterization, effective conductivity may no longer be appropriate. Thus, the linear conductivity of the conductive thread can be used to estimate the effective resistance for design optimization in electronic design automation software such as Advanced Design System (ADS) by Keysight EEsof EDA of Santa Rosa, Calif. Other performance can further be predicted in 3D electromagnetic simulation software such as High Frequency Structure Simulator (HFSS) by Ansys of Canonsburg, Pa. A single coil using electro-textiles such as that shown in FIG. 4B was designed, fabricated and measured. In this particular coil design, Liberator 40 was used as the electro-textile, and silver epoxy was used to connect capacitors, the SMA port and the coil. The coil was simulated near an HFSS 4 mm resolution human neck model before being sewn on cotton cloth.

The performance of the electro-textile RF coil element with the presence of the human neck was also examined. The S₁₁ (reflection coefficient, or return loss) and H₁ (left hand circularly polarized magnetic signal emanated from hydrogen nuclei) of the coil element when conformed to the cylinders of various diameters were further examined. It is noted that the left hand circular polarization notion in MRI literature can differ from that in the field of antennas and propagation, in that it is not defined in the far field region. The propagation direction is replaced with the z-axis, along which the bulk magnetization of the hydrogen nuclei precession occurs. The transmission magnetic field pattern was simulated to show the receive pattern according to the reciprocity theorem.

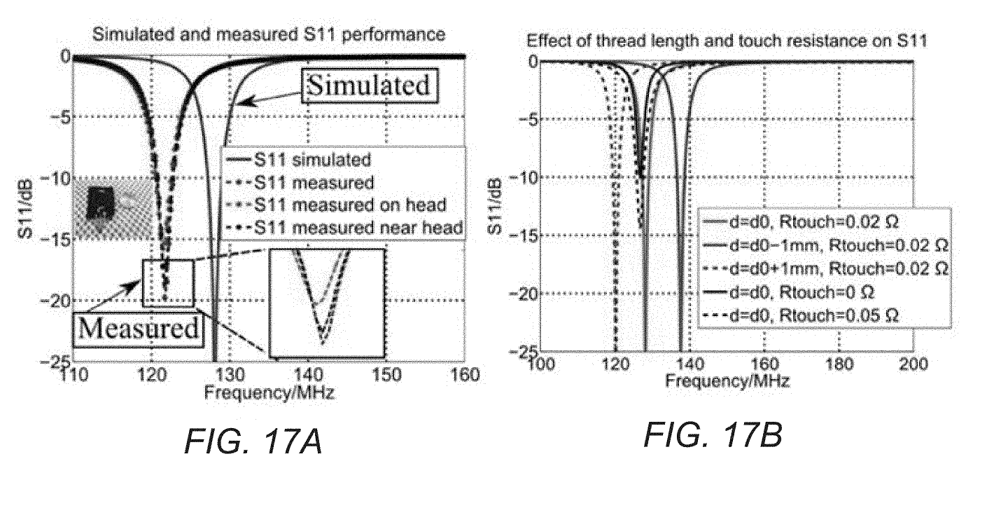

As shown in FIG. 17A, the S₁₁ minimum of the simulated coil near human neck model was −27.5 dB at 128.1 MHz and the measured S₁₁ of the electro-textile coil was −19.8 dB at 122 MHz. Additionally, it was observed that the presence of the human neck did not result in a noticeable change to the resonant frequency. The S₁₁ minimum decreased when the coil was near or on the human neck. It was shown using several versions of the electro-textile coil elements that the slight frequency shift may also be observed depending on the presence or lack thereof of a human neck. Higher effective conductivity and larger coil diameter resulted in more noticeable changes. The noticeable changes of the S₁₁ minimum and the bandwidth was observed in various versions of the prototypes as well. It is noted that the resonant frequency and the S₁₁ minimum is changed by 6 MHz in FIG. 17A. This was mainly a result of the sensitivity of the S-parameter performance to various parameters such as the touch resistance of the capacitors and the electro-textile material, the coil diameter size variation, and the capacitance tolerance. It is shown in FIG. 17B that the resonant frequency was shifted by 10 MHz when the coil element diameter was changed by 1 mm (10% of the original diameter). The S₁₁ was changed from −25 dB to −10 dB when the touch resistance between the capacitor and the electro-textile was changed by 0.02Ω.

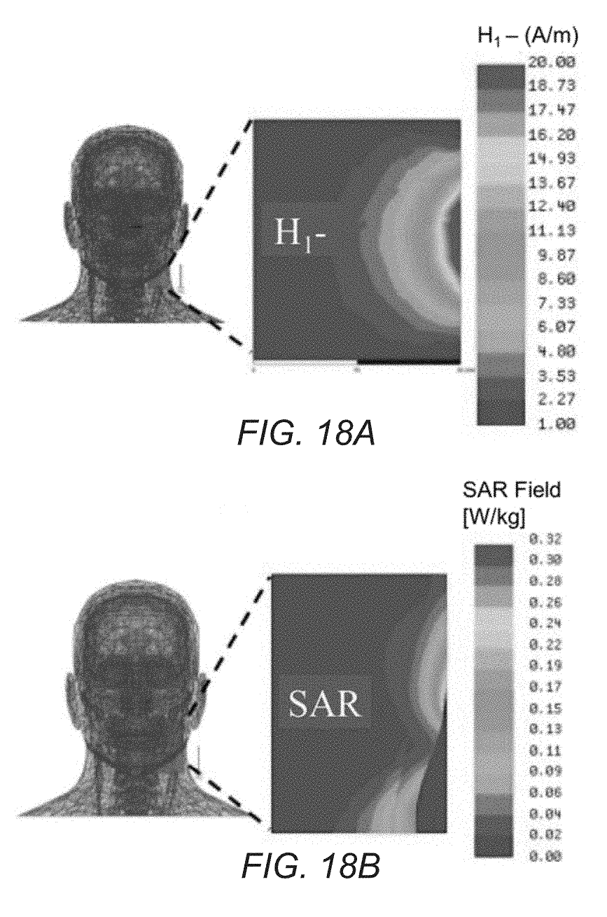

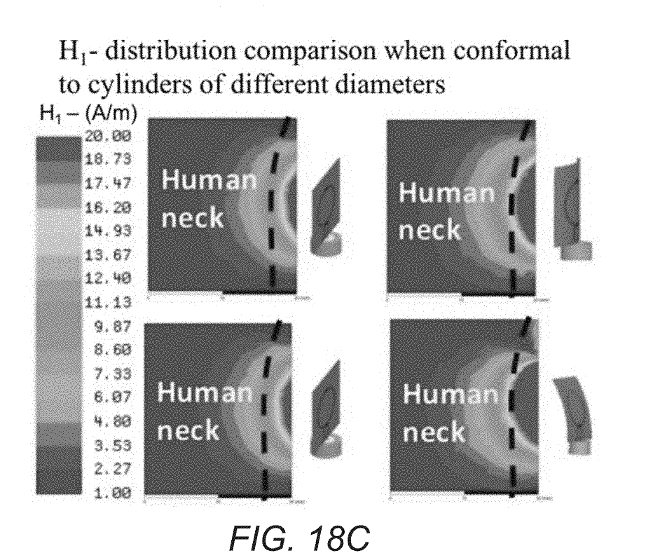

The S₁₁ performance did not, however, exhibit significant change when the coil was conformed to cylinders of different diameters. This is because the resonant frequency is primarily determined by the inductance of the coil and the capacitance of the frequency tuning capacitors, which do not change significantly when conformed to cylinders with reasonable diameters. It was observed that the conformance to cylinders potentially resulted in higher SNR performance than when the coil is flat. The human head model provided by HFSS with minimum voxel length to be 4 mm was used. The H₁ distribution of 30 mm by 35 mm cut 10 mm away from coil center is shown in FIG. 18A. The cut is highlighted along the model's neck. The input power was 1 W, as a reference. Without incorporating a transmission decoupling mechanism, the RF coil elements can also be used as transmission or reception coils when the Specific Absorption Rate (SAR) is important to consider. The SAR performance is also provided in FIG. 18B with a reference input power of 1 W, which is in the same order of magnitude as in real MRI scanner systems. The SAR maximum averaged over 1 g was 0.32 W/kg. The H₁ distribution when the coil was conformed to cylinders of 60 mm diameter is shown in FIG. 18C. The penetration depth was increased when the cylinder diameter was decreased. This observation of potential increase of SNR is also confirmed through the SNR comparison of the MRI images after system integration.

While performance simulation of single-thread test coils is described above with respect to FIGS. 17A-17B and 18A-18C, other simulation methods may be utilized appropriate to the requirements of a specific application in accordance with various embodiments of the invention. Coil array performance in accordance with a number of embodiments of the invention are discussed further below.

### Coil Array Performance

In many embodiments of the invention, RF coil arrays are designed to optimize SNR by minimizing mutual interaction between coil elements. In some embodiments of the invention, two overlapped circular coil elements are designed to achieve low mutual coupling, wherein the mutual flux may be substantially cancelled out. In certain embodiments of the invention, an overlap of approximately 0.48 of the radius is used to obtain the lowest coupling. In a particular example, according to embodiments of the invention, for purposes of illustration and not by way of limitation, a coil element with a 25 mm radius, used in a coil array with a 12 mm overlap, is shown in FIG. 7A. A four-layer structure is used for this prototype of a coil array, with one coil element in the front and the other in the back, as shown in FIG. 7B. A cotton interlayer is used between the two elements. A cotton cushion is also added at the bottom, separating the coil array from direct contact with the human body, as shown in FIG. 7B. The overall thickness of this example array structure is 1.88 mm. The highly conductive electro-textile of Liberator 40 by Syscom Advanced Materials with linear resistance of around 3 Ω/m is used.

This sample RF coil array with electro-textiles was simulated and measured based on the effective conductivity method discussed above in the section regarding performance characterization of electro-textiles. As shown in FIG. 7C for testing, S₁₁ performance was tuned to exactly 127.7 MHz with a minimum of −18 dB, which agrees well with the simulated value. Additionally, the measured S₁₂ had a maximum of around −16 dB, which is within an acceptable range. It was also observed that S₁₁ and S₁₂ would change dramatically if the overlap distance were smaller than 12 mm or larger than 14 mm. The S₁₁ minimum is shifted by 1.1 MHz when a coil of radius 10 mm is conformed to a cylinder of radius 30 mm, which can be tuned back using tunable circuitry. The detailed H₁ (left hand circularly polarized magnetic signal emanated from hydrogen nuclei) simulation with the human model of 4 mm resolution shows that the conformal surface coil penetrates the human body deeper than the flat coils, which implies higher SNR.

While coil array performance is described above with respect to FIGS. 7A-7C, other coil array performance evaluation methods may be utilized appropriate to the requirements of a specific application in accordance with various embodiments of the invention. Coil SNR performance characterization in accordance with a number of embodiments of the invention are discussed further below.

### Coil SNR Performance Characterization

To ascertain a reliable method for characterizing the effects of conductive materials in RF coils on MRI image SNR, researchers performed measurements comparing the performance of coils constructed with conductive cloth, and that constructed with the high-performing standard of copper. One method for predicting the effect of an RF coil on SNR performance is to measure the quality factor ratio with Eq. 4 below. Q_{loaded} and Q_{unloaded} denote the quality factor of the RF coil with and without human loading, respectively. The noise of the RF coil comes from two sources: the human body, and the RF coil material used.

$${Q}_{ratio} = \frac{{Q}_{unloaded}}{{Q}_{loaded}} \qquad ( 4 )$$

Noise from the human body can be primarily associated with the distance from the coil to the human body and the human loading, given a determined coil size. Noise from the RF coil includes the loss from conductive material, substrate material, and the electric components. The Q factor ratio quantifies how dominant the loss is from the human loading among the total loss. It should be noted that the contribution to the loss from the two sources can vary with different coil diameters and frequencies. As a result, a fair comparison of the Q ratio may require a fixed coil size, frequency and measurement setup.

It was measured that copper and conductive cloth versions of the designed RF coil, with diameters of 5 cm, have Q ratios of 2.1 and 1.5 at 127.7 MHz respectively, using the measurement setup that will be discussed in this section. This indicates that the copper coil is less lossy compared with the electro-textile coils as expected.

The relationship between the total available SNR and Q ratio is shown in Eq. 5, where SNR₀ is the total available (or intrinsic) SNR, as described in W. A. Edelstein, G. H. Glover, C. J. Hardy, and R. W. Redington, “The intrinsic signal-to-noise ratio in NMR imaging,” Magnetic Resonance in Medicine, vol. 3, no. 4, pp. 604-618, August 1986, the relevant disclosure of which is hereby incorporated by reference.

$$NR = {SNR}_{0} \sqrt{1 - \frac{{Q}_{loaded}}{{Q}_{unloaded}}} \qquad ( 5 )$$

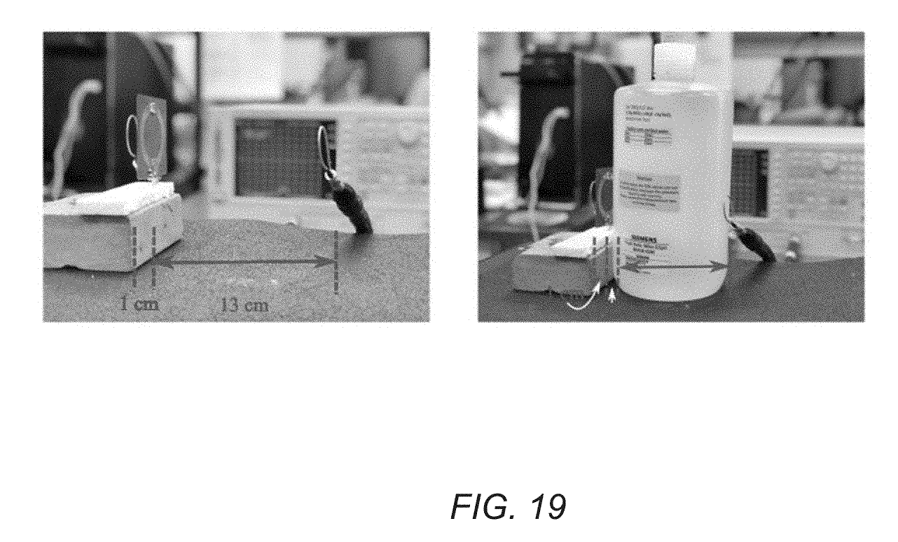

Additionally, it can also be derived that SNR is linearly proportional to \sqrt{Q_{loaded}} under the condition that the same phantom loading is used. As a result, \sqrt{Q_{loaded}} indicates the degree to which the RF coil noise impacts the total available SNR. The performance of RF coils made with electro-textile and copper can be compared by defining relative SNR as in Eq. 6, based on J. R. Corea, P. B. Lechene, M. Lustig, and A. C. Arias, “Materials and methods for higher performance screen-printed flexible MRI receive coils,” Magnetic Resonance in Medicine, vol. 16, pp. 1-9, September 2016, the relevant disclosure of which is hereby incorporated by reference. Utilizing Q_{loaded} measured using the setup in FIG. 19, it is calculated that the relative SNR of the electro-textile copper coil is 40% of that using copper and dielectric substrate.

SNRᵣₑₗₐₜᵢᵥₑ=\sqrt{Q_{loaded}} (6)

The Q factor measurement was conducted at the UCLA Antenna Research Analysis and Measurement (ARAM) lab. The measurement set up is shown in FIG. 19. The two broadband RF probes built at the UCLA ARAM lab were separated by 14 cm and connected with two ports in vector network analyzer HP 8753E. S₂₁ was measured to observe the quality factor of the RF coil being tested. A cylindrical standard phantom was used. The phantom was filled with a 1900 ml solution (per 1000 g H₂O dist.: 3.75 g NiSO₄×6H₂O+5 g NaCl). The conductivity was 1.109 S/m and the relative permittivity was 72.84 at 127 MHz. The RF coil under test was placed 1 cm away from the outer perimeter of the phantom.

While coil SNR performance characterization is described above with respect to FIG. 19, other SNR performance evaluation methods may be utilized appropriate to the requirements of a specific application in accordance with various embodiments of the invention. System integration of RF coil arrays with MRI scanners in accordance with a number of embodiments of the invention are discussed further below.

System Integration of RF Coil Arrays with MRI Scanners

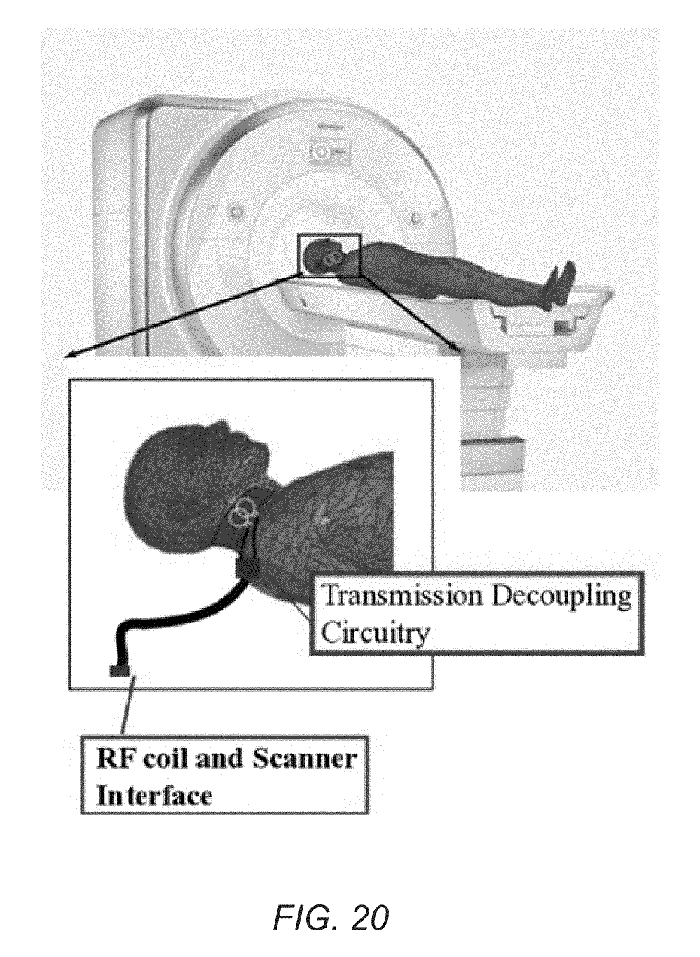

Researchers integrated RF coil arrays formed with conductive electro-textile cloth, according to a number of embodiments of the invention, with MRI scanners and performed test scans. FIG. 20 illustrates a RF coil array system configuration according to an embodiment of the invention, including an RF coil array, transmission decoupling circuitry, and an RF coil interface with MRI scanner. In this system, the RF coil array and transmission decoupling circuitry are connected with coaxial cables. The transmission decoupling circuitry is further connected with the RF coil interface using coaxial cables. The major component of the interface is a preamplifier.

The transmission decoupling circuitry can be used to show high impedance when the MRI scanner is working in the transmission mode. While in the transmission phase, transmission coils typically embedded in MRI scanners are turned on and the hydrogen nuclei are flipped to the xy plane (as shown in FIG. 1). To keep the homogeneity of the static magnetic field and the desired effect by the gradient coils, receive-only coils typically need to be shut down at this time. The transmission-decoupling circuitry can be turned on at this time to shut down the receive-only coil. The coil array and transmission decoupling circuitry can be separated by some distance. This separation can minimize the effect of a high current during transmission mode on the imaging area, and can minimize the device presence to the patient and as a result be more ergonomic.

For performance testing, a conductive electro-textile cloth RF coil array according to certain embodiments of the invention was integrated with a 3T MRI scanner. The images of a homogeneous phantom, a resolution phantom, and a beef phantom were acquired. The images were evaluated quantitatively and qualitatively to analyze the system performance. The conductive cloth coil array was further evaluated by comparing the SNR performance at different depths with the conventional surface coil using the same setup in back-to-back imaging trials.

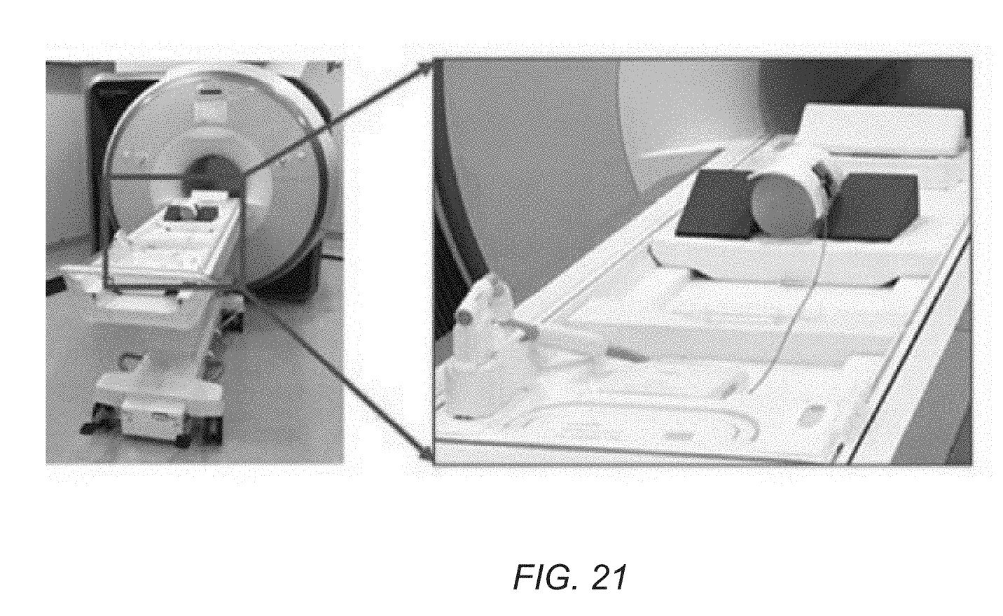

The measurement setup is shown in FIG. 21. The phantom was placed in the position where a human patient's neck would rest. The electro-textile RF coil array with two elements was taped to the phantom with a 1 cm thick cushion in between. The array covered an effective imaging area of 8 cm in height and 5 cm in width, which translates to about 4 cm above and below the mandible angle for an average patient, along with other anatomical structures nearby in the lateral direction.

The images were acquired at UCLA School of Medicine using a 3T Prisma Scanner. A T1 weighted spin echo sequence was applied with a flip angle of 90 degrees, TR=500 ms, TE=20 ms. The field of view (FOV) was 250 mm×250 mm with a resolution of 1 mm×1 mm and a slice thickness of 5 mm.

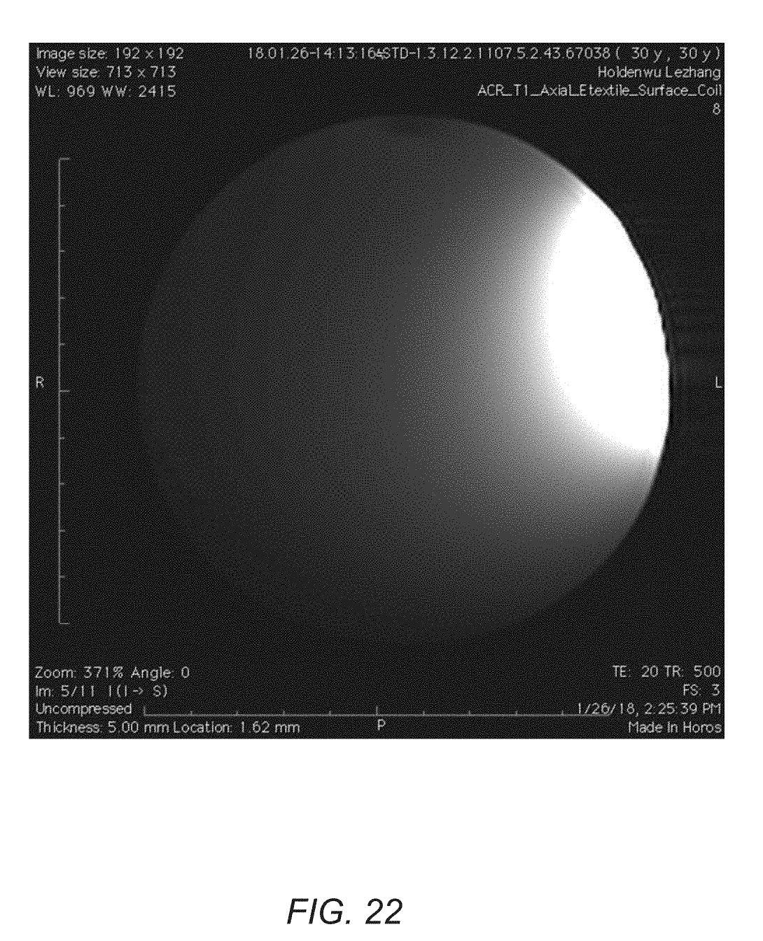

Trials were conducted back to back with the conductive cloth RF coil and a conventional surface coil for each phantom. The image shown in FIG. 22 is the axial plane image of the homogeneous phantom. The ellipse denotes the coil, showing its relative position to the phantom. The slice closest to the MRI scanner isocenter is selected. It can be seen that the signal close to the phantom surface is stronger than at other parts of the phantom. Additionally, the SNR near the surface is higher than that at increased depth. The SNR of the conductive cloth RF coil at 0.5 cm in depth is 4534. By comparison, the SNR of the conventional surface coil at the same phantom depth is 182, after being tightly wrapped around the phantom. The SNR of the conductive cloth coil is 14 dB higher than that of the conventional surface coil in this measurement setup. The SNR of the conductive cloth coil array at a depth of 3 cm is 3 dB greater than that of the conventional surface coil. Further, the SNR of the conductive cloth coil element is 37% of that of the copper coil element of the same size at a depth of 0.5 cm. This result is consistent with the relative SNR approximation of 40% by measuring the loaded quality factor discussed above in relation to coil SNR performance characterization. In contrast to the bare copper coil element used for experimentation purposes, however, the conductive cloth coil array exhibits flexibility for practical use in a medical setting. With the superior SNR performance provided by the system using the conductive cloth coil array, constructed according to some embodiments of the invention, high resolution and contrast of the image is achieved.

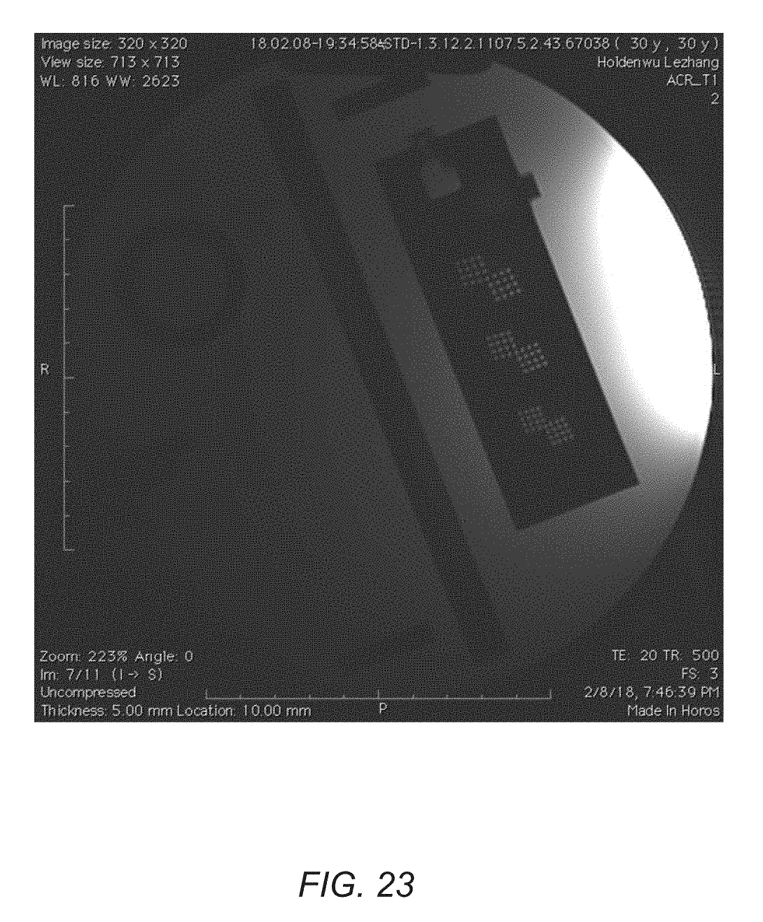

The conductive cloth MRI RF coil array is also used to image a resolution phantom with the same measurement setup and MR sequence as for the homogeneous phantom. In FIG. 23, the ellipse denotes the coil to show its relative position with the resolution phantom. The fine signature of the phantom is marked with a rectangle. Three pairs of squares consisting of 4×4 hole matrix, with the smallest hole dimension being 0.9 mm×0.9 mm, can be clearly observed, although these small features are located at a significant depth. The pixel size is 0.5 mm×0.5 mm. These results indicate reasonable performance with the conductive cloth coil array at greater depths.

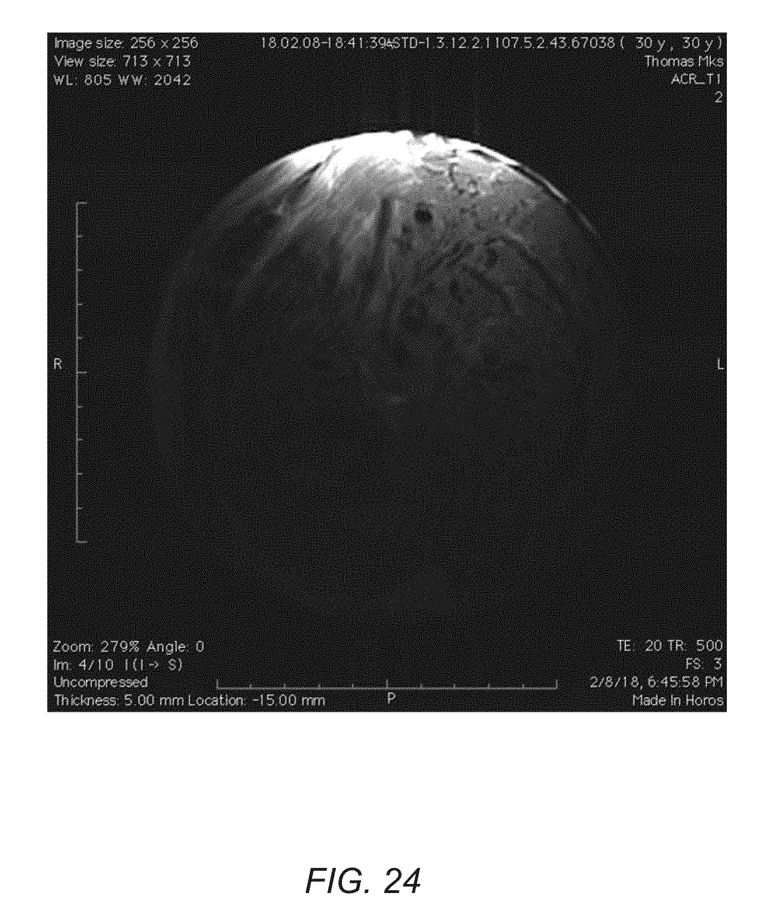

The performance of the conductive cloth RF coil array is further illustrated by imaging a cylinder phantom filled with beef. This beef phantom has a different loading effect as compared with saline water based phantoms. The axial plane image of the beef phantom is shown in FIG. 24. The ellipse denotes the coil to show its relative position with the phantom. The shape and details of the beef can be observed clearly. The parts of the beef phantom with fat (denoted with arrows and circles in FIG. 24) are bright as compared to other tissues at the same depth. These details can still be observed with the overall brightness turned down. The same measurement setups are used here as with the homogeneous phantom.

While system integration of RF coil arrays with MRI scanners is described above with respect to FIGS. 20-24, other system integration and evaluation methods may be utilized appropriate to the requirements of a specific application in accordance with various embodiments of the invention. Applications of electro-textile RF coils in accordance with a number of embodiments of the invention are discussed further below.

### Applications of Electro-Textile RF Coils

Coil elements and arrays according to several embodiments of the invention can be used in various MRI applications to image a variety of medical regions of interest on the human body. In particular, the flexible coils according to many embodiments of the invention are suitable for imaging areas with high curvature, such as but not limited to the neck, knee, elbows, ankles, wrists and other regions to which conventional rigid coils do not closely conform. Furthermore, coil elements and arrays can be utilized in the imaging of non-human subjects. As a specific example and not by way of limitation, a coil array according to certain embodiments of the invention can be used around the neck for carotid artery imaging, such as shown in FIG. 11.

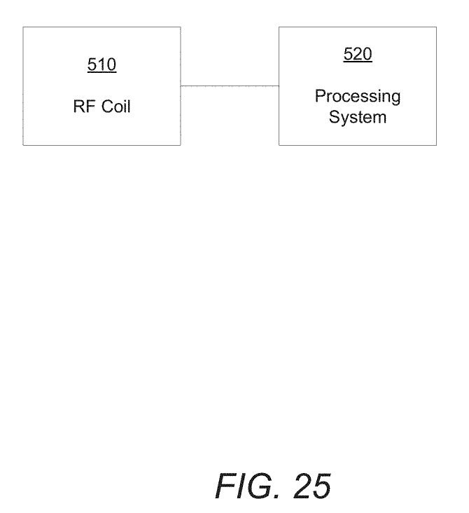

FIG. 25 illustrates an RF coil 510 in communication with a processing system 520 in accordance with an embodiment of the invention. The processing system 520 may be disposed within, coupled to, or independent of a medical device such as an MRI machine. The RF coil 510 may transmit data, such as RF signals received from the patient's body, to the processing system 520.

In many embodiments of the invention, the processing system 520 may store a data processing application to process the data in a variety of ways, including but not limited to generation of MRI images. The processing system 520 may be implemented on a single computing device in accordance with some embodiments of the invention. The processing system 520 may be a personal computer, a laptop computer, and/or any other computing device with sufficient processing power for the processes described herein. The processing system 520 includes a processor, which may refer to one or more devices within the computing device that can be configured to perform computations via machine readable instructions stored within a memory of the processing system. The memory may contain the data processing application as that described above. The processor may include one or more microprocessors (CPUs), one or more graphics processing units (GPUs), and/or one or more digital signal processors (DSPs). According to other embodiments of the invention, the processing system 520 may be implemented on multiple computers.

In some embodiments of the invention, the processing system 520 may include an input/output interface that can be utilized to communicate with a variety of devices, including but not limited to a medical device and/or other display devices. As can be readily appreciated, a variety of software architectures can be utilized to implement a processing system 520 in accordance with several embodiments of the invention.

While various applications of electro-textile RF coils are described above with respect to FIGS. 11 and 25, the coil elements and arrays in accordance with various embodiments of the invention may be utilized as appropriate for a variety of applications.

Although the present invention has been described in certain specific aspects, many additional modifications and variations would be apparent to those skilled in the art. It is therefore to be understood that the present invention can be practiced otherwise than specifically described without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A coil element for magnetic resonance imaging (MRI), comprising:
a fabric;
a coil pattern fixed to the fabric, the coil pattern comprising cut conductive cloth and being capable of receiving radiofrequency (RF) signals from a human body;
a tuning component attached to the coil pattern and capable of adjusting frequency of the coil element; and
an electrical component coupled with the coil pattern, the electrical component capable of transmitting data received at the coil element.

2. The coil element of claim 1, wherein the conductive cloth has a conductivity of at least 10⁵ S/m, at a frequency of 120 MHz.

3. The coil element of claim 1, wherein the conductive cloth has a resistivity between 0.05 Ω/sq and 1 Ω/sq.

4. The coil element of claim 1, wherein the conductive cloth is formed with comprises a non-magnetic and smooth surface.

5. The coil element of claim 1, wherein the electrical and mechanical properties of the conductive cloth are stable at a temperature of at least 200 degrees Celsius.

6. The coil element of claim 1, wherein the coil pattern is fixed to the fabric using by an adhesive.

7. The coil element of claim 1, wherein the tuning component and the electrical component are soldered to the coil pattern.

8. The coil element of claim 1, wherein the tuning component is selected from the group consisting of a varactor and variable capacitor.

9. A coil array for magnetic resonance imaging (MRI), comprising:
a first coil element, comprising:
a first fabric; and
a first coil pattern fixed to the first fabric, the first coil pattern comprising a flexible conductive material and being capable of receiving radiofrequency (RF) signals from a human body;
a second coil element, comprising:
a second fabric; and
a second coil pattern fixed to the second fabric, the second coil pattern comprising a flexible conductive material and being capable of receiving radiofrequency (RF) signals from a human body;
wherein the first and second coil elements are disposed such that the first coil pattern overlaps with the second coil pattern; and
an insulating sheet disposed between the first and second coil elements.

10. The coil array of claim 9, wherein one of the first and second coil patterns comprises conductive thread embroidered into the fabric.

11. The coil array of claim 9, wherein one of the first and second coil patterns comprises cut conductive cloth.

12. The coil array of claim 9, wherein the amount of overlap between the first and second coil patterns is configured to minimize mutual coupling between the first and second coil elements.

13. The coil array of claim 9, wherein the amount of overlap between the first and second coil patterns is configured to maximize signal-to-noise ratio of the coil array.

14. The coil array of claim 9, wherein the insulating sheet is fixed to the first and second coil elements.

15. The coil array of claim 9, wherein one of the first and second coil elements comprises:
a tuning component coupled with the coil pattern and capable of adjusting frequency of the coil array.

16. The coil array of claim 9, further comprising:
a third coil element, comprising:
a third fabric; and
a third coil pattern fixed to the third fabric, the third coil pattern comprising a flexible conductive material and being capable of receiving radiofrequency (RF) signals from a human body;
wherein the first, second and third coil elements are disposed such that the third coil pattern first coil pattern overlaps with at least one of the first and second coil patterns.

17. The coil array of claim 9, wherein the first and second coil elements are configured to conform to an anatomical region of the human body.

18. The coil array of claim 9, further comprising:
a barrier sheet covering one of the first and second coil elements.

19. The coil array of claim 19, further comprising:
a flexible case within which the first and second coil elements are disposed.

20. The coil array of claim 9, wherein the flexible case comprises stretchable material.
